(19) United States
(12) Patent Application Publication
(10) Pub. No.: US 2016/0030476 A1
(43) Pub. Date: Feb. 4, 2016

(54) Compositions, Methods And Devices For Promoting Wound Healing And Reducing Infection

(71) Applicant: IASIS MOLECULAR SCIENCES, Spokane, WA (US)

(72) Inventors: David J. VACHON, Spokane, WA (US); Michael MURPHY, Spokane, WA (US); Scott NORDAHL, Spokane, WA (US)

(21) Appl. No.: 14/776,710

(22) PCT Filed: Mar. 14, 2014
(86) PCT No.: PCT/US14/29762

§ 371 Date: Sep. 14, 2015

Related U.S. Application Data
(60) Provisional application No. 61/785,240, filed on Mar. 14, 2013.

(51) Int. Cl. A61K 33/38; A61K 33/30; A61K 33/34; A61K 45/06; A61K 31/10; C08K 5/3432; A61K 31/444; A61L 15/46; C08K 5/101; C08K 5/435; C08K 5/31; A61K 47/20; A61K 31/155

(52) U.S. Cl. CPC A61K 33/38; A61K 47/20; A61K 33/30; A61K 33/34; A61K 45/06; A61K 31/10; A61K 31/155; A61K 31/444; A61L 15/46; C08K 5/101; C08K 5/435; C08K 5/31; C08K 5/3432; A61L 2300/102; A61L 2300/404

(57) ABSTRACT

Compositions, methods and devices are provided for promoting healing and preventing and treating infection in mammalian subjects. The compositions include pharmacologically active, protease inhibiting, cytokine protecting, aqueous media soluble sulfonated materials, optionally associated with one or more secondary therapeutic agents or carriers, to reduce one or more of inflammation, bacterial proliferation and proteolytic activity. Additionally provided are solubility increasing, stability increasing, toxicity decreasing thiol compounds associated with an antimicrobial compound and optionally secondary therapeutic agents to reduce one or more of inflammation and bacterial infection. Combinations of sulfonated and thiol compounds provide pharmacologically active, protease inhibiting, cytokine protecting, aqueous media soluble, antibacterial, stable, toxicity decreasing, solubility increasing compounds for treating wounds, including burns, in humans and other mammals.

### RELATED APPLICATION

This application claims priority benefit of U.S. Provisional patent application Ser. No. 61/785,240, filed Mar. 14, 2013, the disclosure of which is incorporated herein in its entirety by reference.

### TECHNICAL FIELD

The present disclosure relates to antimicrobial compositions, methods and devices, and to compositions, methods and devices that promote tissue healing in mammals including humans.

### BACKGROUND

Infection remains the most common and serious complication of tissue injuries, as associated with traumatic and surgical wounds, chronic wounds and ulcers, and burns. Infection is particularly problematic following invasive medical procedures and major trauma, or under other circumstances when a substantial portion of the epidermal barrier is damaged. Infection frequently leads to sepsis, which causes approximately 215,000 deaths annually in the U.S. (Natanson C, Esposito C J, Banks S M. The sirens' song of confirmatory sepsis trials: selection bias and sampling error. Crit Care Med 1998; 26(12):1927-31.)

Successful research and development of new antibacterial drugs has fallen steadily since the 1980s, leaving dangerous gaps in current treatment arsenals. Over this same period of innovation decline, persistent overuse of existing antibiotics has led to increased antibiotic resistance among major pathogens, and a concurrent loss of effectiveness of existing drugs to prevent and control infectious disease and sepsis caused by these pathogens. This perfect storm of inaction and misuse has resulted in a precipitous increase of complications and mortality among patients at risk of serious infection.

Normal wound healing involves a complex molecular and cellular response sequence of hemostasis, inflammation, proliferation, and remodeling. Part of this complex response involves a balance of damaged tissue removal, and new tissue formation. Serious infected wounds and chronic wounds fail to exhibit normal healing progression, exhibiting instead abnormalities or defects in one or more mechanisms and stages of healing. Burns or thermal injuries are among the most problematic of wounds, with profound defects in healing attributable in part to an induced state of immunosuppression, predisposing predisposes patients to infectious complications.

Burns are ideal for bacterial growth and provide a rich environment for microbial invasion and growth. This threat increases proportionate to burn surface area. Infection following burns is promoted by loss of the epithelial barrier, presence of dead tissue as a microbial nutrient base, and systemic malnutrition in patients induced by a hypermetabolic response to burn injury. These problems are exacerbated by generalized post-burn immunosuppression, due to an extraordinary release of immunoreactive agents from burn wounds. Sepsis related to burns accounts for roughly 30-65% of burn related deaths. (Mann E A, Baun M M, Meininger J C, Wade C E. Comparison of mortality associated with sepsis in the burn, trauma, and general intensive care unit patient: a systematic review of the literature. Shock. 2012 January; 37(1):4-16. doi: 10.1097/SHK.0b013e318237d6bf.)

Despite the critical importance of sepsis in management of burn wounds, there are only two approved drugs currently employed in the US as topical antibacterial agents for adjunctive therapy against second and third degree burns. One of these approved drugs, silver sulfadiazine, was first described in 1943 by Wruble (M. Wruble, J. Am. Pharm. Ass. 32, 80, 1943) as a mild antiseptic. Silver sulfadiazine was later investigated by Fox (Ch. L. Fox. Arch. Surg. 96, 184, 1968) as a topical treatment agent for burns. While silver sulfadiazine has been reported to be effective against a wide variety of gram-positive and gram-negative organisms, a major drawback of silver sulfadiazine creams is their cytotoxicity, resulting in impairment of re-epithelialization that disrupts or delays healing. Excessive concentration or duration of exposure of wound healing cells and tissues to silver sulfadiazine can negate the antimicrobial benefits of the drug, and these factors are difficult to control or predict. Use of these cream-based products is further complicated by the requirement that the topical coatings be removed (usually by scraping or wiping) prior to application, which causes great discomfort to patents and caregivers. Topical application of creams is naturally attended by a high risk of de novo wound contamination, and thus can only be applied using sterile techniques that are time consuming and typically require a clinical setting.

With the documented rise of antibiotic-resistant organisms, and a critical deficit of approved antimicrobial drugs for treating severe wounds, there is an urgent need for new drugs and treatment methods for preventing and managing wound infection. Effective new antimicrobial agents are needed that can be easily applied to treat and prevent infections, while minimally interfering with normal mechanisms of wound healing.

### SUMMARY OF EXEMPLARY EMBODIMENTS OF THE INVENTION

Disclosed herein are novel compositions and methods for treating and preventing infection and encouraging healing in mammalian subjects, including humans. The compositions described herein can be used for internal or external administration of a therapeutically effective amount of a pharmacologically active antimicrobial compound, complex or formulation. The compositions can be applied directly to a wound as a topical treatment, or can be invested within or attached to a secondary material, matrix, gel, dressing or device. In exemplary embodiments, the compositions contain one or more antimicrobial compounds or agents effective to inhibit bacterial infection, activity and/or growth, while reducing inflammation and promoting wound healing. In certain embodiments, the compositions of the invention also possess protease inhibiting activity, and are thereby effective to minimize adverse impacts of microbial and endogenous proteases, that are particularly damaging in burn wounds. Within these embodiments, the invention provides additional advantages by minimizing destruction or inhibition beneficial cytokines, growth factors and extracellular matrix components in a wound environment, that are essential for coordination of molecular and cellular healing mechanisms. In other exemplary embodiments, antimicrobial compounds and drug agents are provided that are insoluble in deionized water but soluble in ion-comprising aqueous media, affording novel activity and wound healing benefits as described below. These compounds and drug agents may be formulated or cross-linked to alter their solubility or dissolution properties, affording yet additional clinical benefits described.

Antimicrobial compositions of the invention frequently include one or more antimicrobially effective oligodynamic metal(s), such as copper, silver, zinc, and/or bismuth. In more detailed embodiments, an oligodynamic metal is combined with a biologically acceptable thiol compound to decrease toxicity, increase stability, and/or increase absorption of the oligodynamic metal. Thiol compounds may be combined in a delivery formulation with the oligodynamic metal(s), or combined to form an oligodynamic metal-thiol compound or complex, which can be administered internally or externally. Oligodynamic metals may additionally be combined with cationic carriers such as polyacrylic acid, carboxymethycelluloase, alginic acid, and carboxylates, alone or with thiol compound(s), to form antimicrobial compositions for internal and external use.

The novel uses of thiol compounds described herein provides distinct advantages, including by mediating chemical reduction of endogenous thiol compounds, including antioxidants, in a wound. In exemplary embodiments, glutathione attached to an oligodynamic metal provides therapeutic advantages by preserving endogenous glutathione reserves.

In certain embodiments, the compositions of the invention include sulfonated, biologically compatible compounds including polysulfonated compounds. Exemplary sulfonated compounds of the invention include water soluble, anti-protease, cytokine protective compounds. Sulfonated compounds may be employed as a carrier for cationic antiseptics, cationic antimicrobials and/or cationic antibiotics. In certain embodiments, sulfonated compounds are combined with oligodynamic metals. In additional embodiments, sulfonated compounds are combined with a thiol compound, a quaternary ammonium compound, or another antimicrobial compounds such as chlorhexidine or octenidine. In additional compounds, sulfonated compounds are combined with a thiol compound and an oligodynamic metal compound to form novel compounds, conjugates or combinatorial formulations for treatment or prevention of infection, and to promote healing of wounds vulnerable to colonization by microbial pathogens such as bacteria.

In additional embodiments, the oligodynamic metals used herein may be “differentially loaded” (e.g., to provide a selectable concentration, density, or release rate of the oligodynamic metal) within a thiolated, sulfonated, or thiolated and sulfonated, compound, complex, or carrier, to treat different types of wounds (i.e., wounds of different origin, size, status of infection, or stage of healing).

The various active compounds and agents of the invention can alone or together in all combinations as described, and these compounds and agents are additionally employable in combination with secondary, known therapeutic agents, such as conventional antimicrobials, antiseptics, anti-inflammatories, antifungals, growth factors, antioxidants and/or analgesics, for internal or external use. In other embodiments, the antimicrobial compounds, agents and compositions of the invention are useful in combination with other wound therapies, including skin grafting.

In more detailed embodiments, the therapeutic compounds and agents described herein are employed within multistep or multistage treatment protocols to treat and prevent infection and encourage wound healing in mammalian subjects. In exemplary burn management methods, the compositions of the invention enhance wound management through initial application in a high proteinase activity wound stage, where the subject is initially treated intensively with highly anti-proteolytic compounds of the invention described herein. This treatment is maintained until proteinase activity in the subject wound approaches normal levels, at which point the therapeutic compounds with high anti-protease activity are removed and replaced with antimicrobial compounds that do not have high anti-protease activity.

In additional embodiments, compositions of the invention may be formulated in a sustained release formulation, decreasing the need for multiple applications or dressing changes.

In other embodiments, the compositions described herein are activated upon contact with wound exudates (physiological fluids or other ionic solutions), but are not activated by contact with deionized water. This provides novel benefits of inert storage of formulations, dressings and devices bearing active antimicrobial compounds and agents of the invention that can be stored before use and then activated upon application or placement in a wound environment or other therapeutic site.

The foregoing and additional objects and advantages are provided by the invention in the form of novel antimicrobial, healing promoting compounds and agents, methods and devices, as described in further detail below.

### BRIEF DESCRIPTION OF THE DRAWINGS

Exemplary embodiments of the invention are described below with reference to the accompanying drawings, briefly summarized as follows.

FIG. 1 illustrates the effects of Ag⁺ compounds on Pseudomonas aeruginosa growth.

FIG. 2 illustrates the effects of Ag⁺ compounds on Acinetobacter baumannii growth.

FIG. 3 illustrates the effects of Ag⁺ compounds on Klebsiella Pneumoniae growth.

FIG. 4 graphically depicts the effect of silver compound concentration (without reduced glutathione) on cell viability (IC₅₀).

FIG. 5 graphically depicts the effect of silver compound concentration (with reduced glutathione) on cell viability (IC₅₀).

FIG. 6 demonstrates inhibition of elastase by NA-PSS (in comparison to Promogran® (Systagenix, Quincy, Mass.)).

FIG. 7 demonstrates increased inhibition of elastase by NA-PSS in a hydrogel in comparison to gauze or polyester in vacuum assisted closure (V.A.C.) wound fluid.

FIG. 8 is a dose response curve demonstrating inhibition of elastase by SPSS,

FIG. 9 is a graph showing increased inhibition of MMP-9 by Na-PSS formulations in comparison to wound gels alone and gauze.

FIG. 10 is a graph showing increased inhibition of MMP-8 by Na-PSS formulations in comparison to wound gels alone and gauze.

FIG. 11 is a graph showing inhibition of cathepsin G by Na-PSS formulations in comparison to gauze alone.

FIG. 12 is a graph showing inhibition of elastase by Na-PSS formulations in alginate dressing and on beads in comparison to a gauze dressing.

FIG. 13 is a series of graphs showing (a) MMP-9 expression in wounds treated with 8.5% w/w mafenide acetated cream (light bar) vs. control (dark bar); (b) MMP-9 expression in wounds treated with 8.5% w/w mafenide acetated cream and Na-PSS (light bar) vs. control (dark bar).

FIG. 14 provides two graphs showing: (a) Pseudomonas aeruginosa inhibition in wounds treated with 8.5% w/w Mafenide acetate cream for seven days vs. control; and (b) Pseudomonas aeruginosa inhibition in wounds treated with 8.5% w/w Mafenide acetate cream and Na-PSS for seven days vs. control.

FIG. 15 provides infrared spectral results for silver sulfadiazine (SSD), reduced glutathione (GSH and SSD:GSH complex, fingerprint region with SSD as the top line, SSD:GSH as the middle line and GSH alone as the bottom line.

FIG. 16 is a graph showing the half maximal inhibitory concentration (IC₅₀) against human neonatal fibroblasts (dark bars) and 90% inhibition concentration (MIC₉₀) against multi-drug resistant Pseudomonas aeruginosa (Pseudo) (light bars) with therapeutic index IC₅₀/MIC₉₀) above.

FIG. 17 is a graph showing the half maximal inhibitory concentration (IC₅₀) against human neonatal fibroblasts (dark bars) and 90% inhibition concentration (MIC₉₀) against multi-drug resistant Staphylococcus aureus (MRSA)(light bars) with therapeutic index (IC₅₀/MIC₉₀) above.

FIG. 18 is a graph showing the half maximal inhibitory concentration (IC₅₀) against human neonatal fibroblasts (dark bars) and 90% inhibition concentration (MIC₉₀) against multi-drug resistant Acinetobacter baumannii (AcBc) (light bars) with therapeutic index (IC₅₀/MIC₉₀) above.

FIG. 19 is a graph showing the half maximal inhibitory concentration (IC₅₀) against human neonatal fibroblasts (dark bars) and 90% inhibition concentration (MIC₉₀) against multi-drug resistant Klebsiella pneumonia (Kleb) (light bars) with therapeutic index (IC₅₀/MIC₉₀) above.

FIG. 20 is a graph of the therapeutic index for XSC alone and in combination with reduced glutathione (G).

FIG. 21 is a graph of the therapeutic index of PSS-Ag alone and in combination with reduced glutathione (G).

FIG. 22 is a graph of the therapeutic index of SSD alone and in combination with reduced glutathione (G).

FIG. 23 is a graph of the therapeutic index of a comparison of GSH-Ag and PSS-Ag.

FIG. 24 is a graph of the therapeutic index of a comparison of SSD/G and Ascend® Laboratories 1% SSD cream.

FIG. 25 is a graph of the MIC₉₀ comparison between SSD-GSH and SSD alone.

FIG. 26 is a graph of the average inhibition of Pseudomonas aeruginosa, MRSA, Acinetobacter baumannii, Klebsiella pneumoniae, and Escherichia coli using co(poly(divinyl benzene)-poly(styrene sulfonate) reacted with varying amounts of cysteine or glutathione.

FIG. 27 is a graph of the biocompatibility of gel formulations of different therapeutic compounds of the invention over time.

FIG. 28 is graph of the effectiveness of gel formulations of different embodiments of the invention over time against Pseudomonas aeruginosa.

FIG. 29 is a graph of the effectiveness of gel formulations of different embodiments of the invention over time against MRSA.

### DETAILED DESCRIPTION OF EXEMPLARY EMBODIMENTS OF THE INVENTION

Disclosed herein are novel compositions and methods for preventing or treating infection and promoting healing in mammalian subjects. The compositions may be applied directly to a wound or may be formulated in a topical gel, cream or other carrier, or attached to a wound dressing or other device. The compositions, methods and devices of the invention reduce or prevent bacterial infection and proliferation, and promote/accelerate wound healing. In certain embodiments the compositions of the invention are effective to reduce proteolysis and protect healing biomolecules (e.g., cytokines and growth factors), and otherwise reduce adverse inflammatory events in a wound environment, further promoting wound healing.

In certain embodiments, the therapeutic compositions described herein include one or more oligodynamic metals, alone or in combination with one or more biologically acceptable thiol compounds. The combination of a thiol compound with an oligodynamic metal within the methods and compositions of the invention affords unexpected advantages of decreased toxicity, increased stability, and increased absorption or bioavailability of the oligodynamic metal in a wound environment. Thiol compounds that find use within the invention are effectively combined in a delivery formulation with the oligodynamic metal(s), optionally in the form an oligodynamic metal-thiol compound or complex, that may be formulated for internal or external administration. Oligodynamic metals may additionally be combined with polyacrylic acid, carboxymethycelluloase, alginic acid, and carboxylic acids, alone or with thiol compounds, to form therapeutic antimicrobial compositions for internal and external administration.

In other embodiments, the compositions described herein may be sulfonated biologically compatible compounds. Exemplary sulfonated compounds for use within the invention include water soluble, anti-protease and cytokine protective compounds. The sulfonated compounds may further operate as a carrier for cationic antiseptics and/or cationic antibiotics. In certain embodiments, the sulfonated compound may be a polysulfonate polymer. In additional embodiments, the sulfonated compounds may be combined with thiol compounds, quaternary ammonium compounds, or other antimicrobial compounds such as chlorhexidine or octenidine, to form novel compounds for the treatment or prevention of infection and promotion of wound healing.

The compounds, compositions, methods and devices of the invention may be used alone or in combination with secondary therapeutic agents including, but not limited to, antimicrobials, antiseptics, antifungals, growth factors, antioxidants and/or analgesic agents suitable for internal or external administration to a mammalian subject.

Compositions of the invention may incorporate one or more antimicrobial compounds or drugs described herein formulated in oil-in-water emulsion, polymer matrices, creams, ointments, lotions, amorphous hydrogels (gels), or non-amorphous hydrogels, for application to a wound or other microbially vulnerable healing compartment, to treat or prevent of infection while promoting healing. The compounds and formulations may be applied directly to a wound surface, or may be integrated with a carrier or device such as a biogel film, patch, dressing, bandage, wound covering, or other useful device for biomedical drug application. In some embodiments, the therapeutic compounds may be employed within a multi-step treatment process, with a first step comprising one or more applications of an anti proteolytic, cytokine protective compound, and a second step comprising one or more applications of an antimicrobial therapeutic compound without substantial anti proteolytic or cytokine protective activity. Formulations of the invention may optionally include one or more secondary therapeutic agents such as antimicrobials, antiseptics, antifungals, growth factors, antioxidants and/or analgesic agents.

In more detailed aspects of the invention, addition of a biologically acceptable thiol improves color stability and cosmetic impact of the formulation, while improving effectiveness of the active agent (including through increased solubility) and decreasing toxic effects on mammalian cells due to exposure to the active agent. The novel use of a thiol in this context yields particularly surprising results. Because efficacy of the anti-microbial agent and its salts against bacteria is thought to result from disabling interactions of the anti-microbial with a native sulfhydryl moiety within proteins on the surfaces of bacterial cell walls, the improved effectiveness of anti-microbial formulations containing exogenous sulfhydryl or other thiol compounds is counterintuitive.

The addition of sulfonated groups, including water soluble natural, semisynthetic, or synthetic sulfonated polymers imparts anti-protease and cytokine protective activities to the compositions, promoting wound healing and arresting the destruction of healthy tissue often seen in major trauma and severe burns.

### DEFINITIONS

Topical medication refers to a medication applied to body surfaces, such as skin or mucous membranes. Topical medications of the invention incorporate the active antimicrobial or other drug agent(s) in a topical delivery carrier, including but not limited to polymer matrices, creams, foams, gels, lotions and ointments (including include oil-in-water emulsions).

The term “suspended” refers broadly to a non-dissolved “dispersion” of active material (e.g. silver sulfadiazine) in a base liquid or semi-solid carrier. The active material is preferably finely divided and dispersed homogeneously throughout the base formulation.

The term “aqueous solution” refers to a liquid mixture containing water.

The term “solvent” refers to a liquid capable of dissolving a substance. A “water-miscible solvent” is capable of being stably mixed with water.

Carboxylates as used herein include salts or esters of a carboxylic acid. In the salt form, a carboxylate anion may be paired with metallic or organic cations.

Sulfonates can be salts or esters of a sulfonic acid. The sulfonate functional group is represented as R—SO₂O⁻, and may paired with a metallic or organic cation.

The term “wound” includes a burn, cut, sore, blister, ulcer, rash or any other lesion or area of disturbed skin that generally involves breach of the dermal protective layer.

The terms “microbe” and “microbial” refer to bacteria, fungi, and viruses. The terms “antimicrobial” and “antimicrobial activity” refer to the ability to kill or inhibit the growth of microbes, particularly pathogenic microbes capable of colonizing wounds and adversely affecting host systems and healing processes.

The term “photostable” means that an object or material is resistant to discoloration when exposed to ambient light.

The terms “matrix”, “matrices” and the like refer broadly to materials in which antimicrobial compounds and agents, such as silver species, can be embedded, attached, dispersed, or otherwise associated with. A “polymer matrix” is one type of matrix comprising one or more natural or synthetic compounds, usually of high molecular weight, comprising repeating units or monomeric components. Examples of polymer matrix materials for use within the invention include, but are not limited to, sodium carboxymethyl cellulose, pectin, gelatin, polysaccharides (alone or mixed with other matrix materials), and ion exchange materials (strong and weak forms). In certain embodiments the polymer matrix is a sulfated polysaccharide (polysulfated polysaccharide) such as heparin, heparan sulfate, chondroitin sulfate, keratan sulfate, dermatan sulfate and the like. Other sulfated glycosaminoglycans and polycarboxylated materials, including carboxymethylcellulose, alginic acid, and hyaluronic acid (hyaluronan), and other carboxylated polysaccharides (sugar acids), are readily adapted for use within the invention.

The term “hydrogel” may refer to an amorphous hydrogel, typically comprised of non-fixed, three-dimensional macrostructures hydrophilic polymers or copolymers. A hydrogel may alternatively comprise polymers or mixtures of polymers that maintain solubility in water, or non-amorphous hydrogels (fixed macrostructures) that swell, but do not dissolve in the presence of water. Hydrogels may or may not be cross-linked, which property can be selected and adjusted as a means to limit and meter delivery of antimicrobial compounds in cross-linked or partially cross-linked hydrogels.

### Compositions and Methods for the Treatment of Wounds

Wound healing is a complex process where the body repairs itself after injury. Generally, wound healing involves hemostasis, inflammation, proliferation and remodeling. The process is fragile and susceptible to interruption or failure due to infection, diabetes, venous or arterial disease, or metabolic deficiencies. Burn wounds can be of a variety of origins, including thermal, chemical, electrical and radiation insults damaging and disrupting the protective elements of the skin. Burn wounds are readily colonized by bacteria and other microbial pathogens that impair wound healing and can result in life threatening septicemia if untreated. Burn wounds are further complicated by immunosuppression that predisposes burn patients to infectious complications. Burn patients are at additional risk for related complications of sepsis secondary to pneumonia, catheter-related infections, and suppurative thrombophlebitis.

Local inflammation following injury is essential for wound healing and host defense against infection. However, trauma and burns of severe magnitude and degree can induce overwhelming inflammatory responses, causing a host of adverse impacts ranging from local tissue destruction to systemic inflammatory response syndrome and septic shock (resulting in cellular, tissue and even organ destruction). Hyper-inflammatory responses in burn wounds are often associated with healthy tissue loss at the site of the wound, leading to increased risk of infection and increased healing time.

Many mechanisms of wound healing require a delicate balance between oxidative stress and antioxidants. Exudates from wounds, particularly severe or non-healing wounds, contain elevated levels of proteolytic enzymes like elastase from polymorphonuclear granulocytes (PMN elastase), reactive oxygen species (ROS) and reactive nitrogen species (RNS). The overproduction of proteinases in wounds causes reduced concentrations of active growth factors and proteinase inhibitors, triggering an imbalance between degradation and remodeling processes that delays wound healing.

Matrix metaloproteinases (MMPs) such as MMP-9 are transiently expressed in normal wound healing, but may be adversely elevated in chronic wounds and in cases of prolonged inflammation. Elevated levels of MMP-9 interfere with tissue remodeling, delaying healing and preventing migrating keratinocytes from forming new attachments to newly synthesized basement membrane (M J Reiss, MD, Y P Han, PhD, E Garcia, MD, M Goldberg, MD, Y K Hong, PhD, and W L Garner, MD Matrix Metalloproteinase-9 Delays Wound Healing in a Murine Wound Model Surgery. February 2010; 147(2): 295). In order for healing to progress and result in repair, a balance must be maintained between protein-degrading activities of MMPs and other cellular activities directed toward synthesis and deposition of protein components of granulation tissue. MMP-9 is also believed to be involved in recruitment of neutrophils to active wound healing sites.

Neutrophils are considered to be the primary cell for cleaning microorganisms from wounds. If there is excessive neutrophil activity due to high bacterial counts, the byproducts of neutrophils can negatively affect the wound tissue and damage healthy tissue. In chronic wounds, a protracted inflammatory response is mediated by the continued presence of inflammatory leukocytes, most notably neutrophils. Neutrophils also release serine proteases such as elastase. Though all wounds require a certain level of elastases and proteases for proper healing, excessive concentrations of these agents impair wound healing. Elastase increases inflammation, destroys tissue, proteoglycans, and collagen, and damages growth factors, fibronectin, and factors that inhibit proteases.

The therapeutic compounds described herein are uniquely useful to regulate proteolytic activity in wound healing environments, and in certain embodiments are adapted for staged application to differentially regulate proteolytic activity in wounds. In certain embodiments, anti-proteolytic compositions and methods of the invention are employed during early stages of wound healing to reduce excessive proteolytic activity during these stages. Anti-proteolytic activity of the inventive compositions is selectable and metered to reduce anti-proteolytic activity in later stage wound healing, to maintain an optimal balance between protein degrading activities and protein synthesis to facilitate wound healing. By limiting early stage hyperinflammatory responses, including elevated proteolysis, these novel compositions and methods also mediate protection of cytokines, growth factors and other biomolecules essential to optimize healing responses.

Common burn wound pathogens such as Pseudomonas aeruginosa produce a number of cell-associated (adhesins, alginate, pili, flagella, and lipopolysaccharide) and extracellular (elastase, exoenzyme S, exotoxin A, homolysins, iron-binding proteins, leukocidins, and proteases) virulence factors that mediate a number of processes, including adhesion, nutrient acquisition, immune system evasion, leukocyte killing, tissue destruction, and bloodstream invasion leading to an interruption and dysregulation of the wound healing process.

Antioxidants such as the thiol containing glutathione protect protease inhibitors from oxidative damage, helping to regulate the presence of proteases in the wound healing process. Thiol groups are reducing agents, normally existing at a concentration of approximately 5 mM in animal cells. Thiol antioxidants act through a variety of mechanisms, including (1) as components of the general thiol/disulfide redox buffer system, (2) as metal chelators, (3) as radical quenchers, (4) as substrates for specific redox reactions (GSH-reduced glutathione), and (5) as specific reductants of individual protein disulfate bonds (thioredoxin) (Deneke S M. Thiol-based antioxidants. Curr Top Cell Regul. 2000; 36:151-80). For example, glutathione reduces disulfide bonds formed within cytoplasmic proteins to cysteines by serving as an electron donor. It is the major endogenous antioxidant produced by the cells, participating directly in the neutralization of free radicals and reactive oxygen compounds, as well as maintaining exogenous antioxidants such as vitamins C and E in their reduced (active) forms. In the case of severe trauma such as burns, serum GSH levels are significantly reduced in comparison to healthy subjects (A. S. Sahib, F. H. Al-Jawad, and A. A. Alkaisy, Effect of Antioxidants on the Incidence of Wound Infection in Burn Patients, Ann Burns Fire Disasters. Dec. 31, 2010; 23(4): 199-205).

In some embodiments, the compositions described herein combine antimicrobial oligodynamic metals with thiol compounds. In other embodiments, the compositions described herein combine antimicrobial oligodynamic metals with sulfonate groups. In further embodiments, the compositions described herein combine antimicrobial oligodynamic metals with thiol and sulfonate compounds. In other embodiments, the compositions described herein combine quaternary ammoniums with thiol compounds or sulfonate compounds or both. In additional embodiments, oligodynamic metals may be delivered to wound sites using polyacrylic acid, carboxymethyl cellulose, alginic acid, and carboxylate. In some embodiments, the sulfonate groups may be sulfonated polysaccharides including, but not limited to, heparin sulfate, chondroitin sulfate, dermatan sulfate, keratin sulfate, and aggrecan sulfate. The compositions described herein have antimicrobial activity and are not subject to the antibiotic resistance developing for many antimicrobial compounds. Additionally, sulfonated compounds as described herein have anti-protease activity and growth factor protective activity allowing protease levels to be regulated as required for the healing process. These therapeutic compounds may additionally be combined with secondary therapeutic agents including, but not limited to, antimicrobial, antiseptic, antifungal, growth factors, antioxidant and/or analgesic agents.

The addition of thiol groups to antimicrobial compounds within the invention increases solubility and decreases toxicity of antimicrobial oligodynamic metals. Thiol-containing compounds and formulations of the invention are provided which increase the solubility of oligodynamic metals by as much as 50%, up to 2 fold, 3 fold, 4 fold, 5 fold, 6, fold, 7 fold, 8 fold, 9 fold, 10 fold, or even higher, compared to the same compounds and formulations lacking thiol. Thiol addition within the formulations of the invention concurrently decrease toxicity of oligodynamic metals to endogenous wound healing cells. Compared to thiol-minus compositions, thiol-containing compounds and formulations exhibit reduced toxicity to mammalian cells operative in wound healing by as much as 20-25%. 30-50%, 50-75%, 75%-90% or greater compared to toxicity observed in the absence of added thiol. Expressed alternatively, toxicity observed in control assays exposing cells to oligodynamic metals alone, may be 2-3 times greater, 4-5 times greater, 6-10 times greater, 20 times greater, 30-40 times greater, and even 50-75 times greater than toxicity levels observed in thiol-added compounds and formulations of the invention.

Additional unexpected advantages of the invention are revealed in the context of endogenous glutathione metabolism. Addition of glutathione to the antimicrobial compounds of the invention surprisingly prevents reduction of endogenous glutathione and glutathione transferase reserves (despite the presence of an oligodynamic metal, such as silver, which generally inactivates glutathione transferase and taxes native glutathione reserves). The addition of glutathione in combination with the antimicrobial compounds of the invention uniquely prevents reduction of endogenous glutathione and glutathione transferase. In more detailed embodiments, glutathione transferase activity (and/or endogenous glutathione levels) is/are elevated in the presence of added thiol (e.g., glutathione) formulations by at least 10%, 15-20%, 20-30%, 30-50%, 50-75% or higher, compared to levels observed following administration of the antimicrobial compound alone (e.g., in in vitro assays, or as measured in post-treatment wound exudates.

Oligodynamic metal compounds useful within the invention include, but are not limited to, mono and divalent compounds such as copper, silver, zinc, and bismuth in all of their forms. Exemplifying these subjects, a silver component of the composition may be one or more of a silver halide, silver nitrate, silver sulfate, silver monocarboxylate, silver oxide, silver salt of a sulfonamide, silver salt of a polycarboxylic acid, silver particles, silver salt of a polysulfonic acid, silver salt of a polysulfated polysaccharide, a silver phosphate, a silver carbene, an organosilver compound, or silver metal. All of these oligodynamic metal compounds have antimicrobial properties, but with differing properties of bioavailability, antimicrobial efficacy and toxicity to mammalian cells. Considering that Ag⁺ alone is active against over 600 different types of bacteria, fungi and viruses, silver-based antimicrobial agents are of particular interest within the invention. However, there are complex issues concerning the toxicity of silver to mammalian systems which have heretofore limited the use of silver in antimicrobial and wound healing applications. Additionally, silver is associated with strongly disfavored skin discoloration and irritation in patients (as is widely associated with the use of silver nitrate). Absorption of silver evinced by systemic distribution and excretion in urine has also been reported.

Carriers of oligodynamic metal compounds within the compositions of the invention include various thiol compounds, polyacrylic acid, carboxymethycelluose, alginic acid, and carboxylates, among others, which allow favorable delivery of antimicrobial metals to wound. The use of thiol compounds notably decreases toxicity of the oligodynamic metals. In exemplary embodiments, silver compounds alone are 50-100% more toxic, 2-3 times more toxic, 5-10 times more toxic, up to 20 times and even 50 times more toxic or greater compared to reduced toxicity observed when silver is combined in a novel thiol-containing combinatorial compound or composition of the invention.

Combining oligodynamic metal compounds with thiol groups according to the teachings herein increases the solubility of the oligodynamic metal (rendering the metal 2-3 times more soluble, 5-10 times more soluble, 10-100 times more soluble, 100-1,000 times more soluble, up to 1,000-10,000 times more soluble or greater) in physiological fluids (e.g., wound exudates). This allows more of the anti-microbial metal to be delivered to sites of wound healing. In exemplary embodiments, silver coupled with a thiol increases solubility of silver by up to 10,000 times or greater than the solubility of silver in the absence of thiol.

Thiol compounds useful within the compositions and methods of the invention include, but are not limited to, glutathione, penicillamine, bacillithiol, mycothiol, cysteine, 4-mercaptophenylboronic acid (as described in WO 2013120532, incorporated herein by reference), and acetylcysteine. According to novel methods for producing antimicrobial compositions herein, thiol compounds may be combined with a desired antimicrobial cation to form, e.g., an oligodynamic metal-thiol combinatorial compound. For example, reduced glutathione (GSH) may be reacted with a cation-acetate salt as follows:

GSH-AgOAc→GSH-Ag+HOAc

Generally, glutathione is solubilized in deoxygenated deionized water. A mass of acetate salt sufficient to obtain a quantitative loading of the desired cation is added. The mixture is then stirred with a stir-plate or overhead mixer until the acetate metal salt is fully solubilized or evenly suspended if the compound exhibits low solubility. The mixture is then frozen at −80° C. The frozen mixture is lyophilized and the resulting product is isolated and ground to desired fineness. These novel preparations and their combined starting materials and intermediates represent operable embodiments of the invention.

In alternate methods of producing antimicrobial preparations, thiol compounds may acquire cations from non-acetate salts. For example, GSH may be solubilized in deoxygenated deionized water. A mass of non-acetate salt sufficient to obtain a quantitative loading of the desired cation is then added to the vessel containing the GSH solution. The mixture is then stirred with a stir-plate or overhead mixer until acetate metal salt is fully solubilized or evenly suspended if the compound had low solubility. The mixture is then frozen at −80° C. The frozen mixture is lyophilized and the resulting product is isolated and ground to desired fineness.

In certain embodiments of the invention, differentially active and specifically useful compositions are prepared by “differential loading” or “metered loading” of an antimicrobial cation in combination with a thiol compound. In exemplary aspects, the cation-thiol combinatorial compound may contain a selected loading of 1%, 10%, 20%, 30% 40%, 50% or more of a cation such as silver by percent substitution (e.g., percent of total Na+ or other available substitution targets) or by weight (e.g., % w.w. of a silver-thiol compound, or % w.w. of a silver-thiol loaded polymer). The amounts of cation such as silver attached to the thiol compound may be adjusted depending on the use for the compound. For example, for early wounds at high risk of infection a high percentage (e.g., 20%, 30-50% or greater) of silver loading may be used, while in later stages of wound healing a lesser concentration (e.g., below 30%, 10%, or lower) of the anti-microbial may be used, allowing the normal healing process to take over.

In addition to combinatorial compositions with thiol groups, enhanced delivery and performance of oligodynamic metals in wounds can be achieved using a novel carrier exemplified by polyacrylic acid, carboxymethycelluose, alginic acid, and carboxylate. These carriers may be multi-target and multi-functional, in that they may also be employed to carry cationic antiseptics, antibiotics, antifungals, growth factors, anti-inflammatories, analgesics and anesthetics, among other secondary therapeutic agents.

In additional synthetic methods of the invention for making antimicrobial compositions, acetate salts may be used to attach cations such as oligodynamic metals to carboxymethyl cellulose sodium (CMC-Na) or alginic acid using the following scheme generalized. CMC-Na or alginic acid is solubilized in deoxygenated deionized water. To a vessel containing the CMC-Na or alginic acid solution, a mass of acetate salt sufficient to obtain a quantitative loading of the desired cation is added. The mixture is then stirred with a stir-plate or overhead mixer for 30 minutes at room temperature (˜20° C.) until a strong acetic acid smell is evident. The solution is then added drop wise to 2 L of 100% alcohol (reagent grade 90% ethanol, 5% methanol, 5% isopropanol) under high shear and stirred for 5 minutes. The precipitated polymer is filtered under vacuum and the solid washed 4× with 100% alcohol until no odor is detectable. The precipitated product is then collected and dried in a vacuum oven for 4-18 hours at 60° C. The functionalized CMC-X or alginic-X is stored in a dry amber or opaque bottle at or below 20° C.

In addition to compositions with a selectable combination of members optionally including thiol groups, polyacrylic acid, carboxymethycelluose, alginic acid, and carboxylate groups, the compositions described herein may include pharmacologically active, protease inhibiting, aqueous media soluble polysulfonated materials in salt forms, in a liquid or solid mixture to reduce one or more adverse activities in a wound healing environment selected from inflammation, proteolytic activity, bacterial proliferation, and cancerous cell growth. Sulfonated compositions combinatorially useful within the invention may also exhibit marked cytokine protective activity. Sulfonate compounds alternatively useful within the compositions and methods of the invention include, but are not limited to, polysulfonated polymers, sulfadiazines, polysaccharides, polyvinyl sulfates, poly acrylamidomethyl propane sulfates, poly methyl styrene sulfonates, poly(methyl styrene sulfonate)-co-(polymethylmethacrylate)s and poly anethole sulfonates.

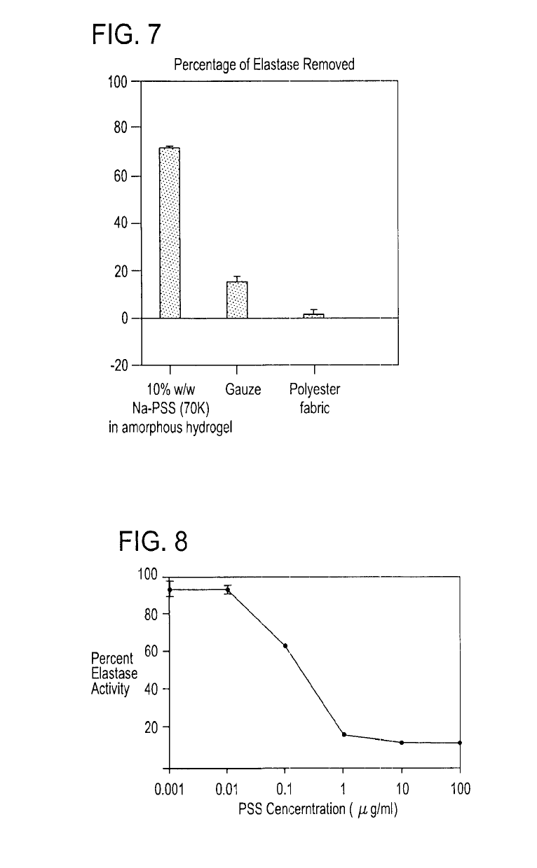

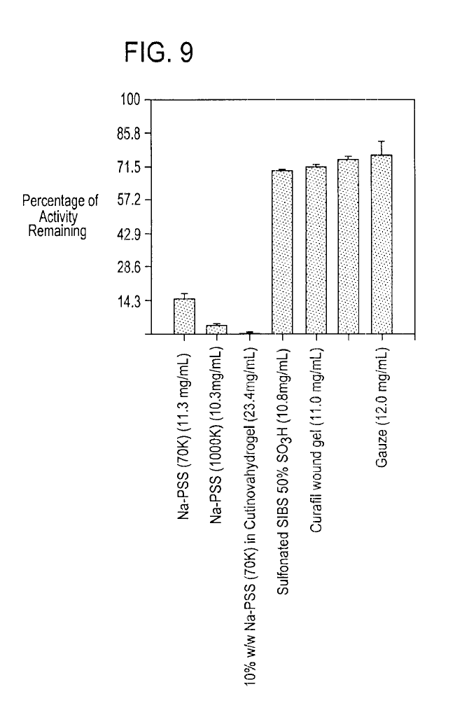

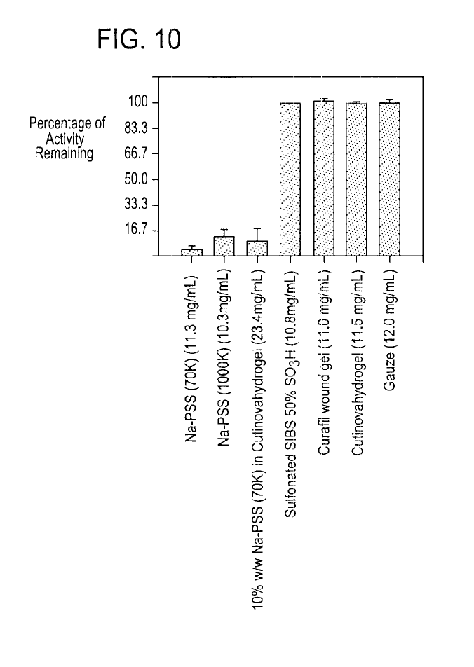

The polysulfonated therapeutic compounds described herein are natural, semisynthetic, or synthetic polysulfonated proteinase inhibitors of both high isoelectric point proteinases and metalloproteinases. For example, as shown in FIGS. 9 and 10, water soluble polysulfonated compounds such as sodium polystyrene sulfonate (Na-PSS) effectively inhibit proteinases MMP 8 and MMP 9. Additionally, as shown in FIGS. 6 and 7, Na-PSS also inhibits elastase.

Yet another novel and surprising feature of the invention follows the discovery that solubility of polysulfonated materials described herein can be tailored to be render formulations that are effectively insoluble in deionized water, yet soluble in ionic aqueous media (e.g., physiological solutions, including wound exudates). This provides extraordinary advantages by allowing easier application and controlled storage and release/activation of the antimicrobial compositions into a wound environment upon contact.

Polysulfonated compounds as described herein can be a polysulfonated material, a polysulfated material and/or a polysulfonic acid salt or a polysulfated material of acid or salt form thereof. In some instances, the repeating unit of the polysulfonated compound may be represented chemically as [R′(SO₃⁻X⁺)m]n with m representing the number of sulfonates or sulfates within a repeating unit of a macromolecule and where n is at least one and m is greater than 1. The R group contains carbon, hydrogen, and may possess other atoms including heteroatoms such as nitrogen, sulfur, and oxygen. “X” can be one or more variable cations including metal and organic species and may comprise mixtures of metal cations including the oligodynamic metals described herein, organic cations, or mixed metal cation-organic cation combinations. When the R′ group of the polysulfonated compound is a repeating unit of a polymeric material, for example a polysaccharide such as a glycosaminoglycan, it is understood that the R′ group possesses an oxygen atom that is covalently linked between the ring (carbon) of the sugar and the SO₃⁻X⁺ functionality. As such, it is understood that sulfonate groups of the sulfonated therapeutic agents are associated with either counter cations or protons (H+), in order to maintain nature's law of neutrality.

The R′ group can be the backbone of an oligomer, such as a dimer and/or trimer, or a polymer. The oligomer or polymer can comprise monomeric units of arylenevinyl sulfonate, styrene sulfonate, alkyl styrene sulfonate such as methyl styrene sulfonate, sulfated saccharides, and/or vinyl sulfonate monomers as well as nonsulfonated monomers. The oligomer can include repeating units of the same monomer or more than one monomer where the monomer may be chiral, achiral or a racemate.

The polysulfonated therapeutic compounds described herein can also include other sulfonated compounds such as, but not limited to, polymers of sulfated saccharides or polysulfated polysaccharides, such as dextrin sulfate, dextran sulfate, chitosan sulfate, or cellulose sulfate, among others. The sulfated polysaccharide may be a proteoglycan with main chain components that include a dermatan sulfate, or a keratan sulfate, among others. Proteoglycans useful within the invention additionally include, but are not limited to, aggrecan, versican or smaller sized species that include decorin, biglycan, fibromodulin, keratocan, osteoglycin, and lumican, among others.

The sulfonate group of the therapeutic compounds and compositions of the invention can be coupled directly to a structural unit depicted by —OR″, with the R″ group representing the remainder of the polysulfonated therapeutic compounds, and the coupling to an oxygen atom {O} forming what is referred to as a sulfate group (—OSO₃⁻X₂⁺). Accordingly, sulfate groups contain sulfonate groups (—SO₃⁻X₂⁺). As such, the polysulfonated therapeutic compounds can include polysulfonates including sulfonic acids, sulfonic acid salts, poly(vinyl sulfonate) derivatives, poly(methyl styrene sulfonate) derivatives, poly(methyl styrene sulfonate) derivatives, poly(methyl styrene sulfonate)-co-(polymethylacrylate) derivatives, and poly(anethole sulfonate) derivatives among others.

Polysulfated compounds for use within the invention can include synthetic, semi-synthetic, and/or naturally occurring polysulfated polysaccharides that include chondroitin sulfate, dermatan sulfate, keratin sulfate, heparan sulfate, heparin, or dextran sulfate given as an example above, as well as the sulfated semisynthetic polysaccharide pentosan polysulfate. Sulfated polysaccharides (also known as glycosaminoglycans or GAGs) are efficient ion exchange materials by virtue of the sulfonate group present.

In some cases, the polysulfonated therapeutic compounds can have a molecular weight of from about 600 grams/mole to about 1,000,000 grams/mole but may be in excess of 2,000,000 g/mole.

Oligodynamic metal analogs of polymers such as polysulfonates, polycarboxylates and polyphosphorylated compounds may be formed by a variety of methods. In certain embodiments, the acid form with a pKa<4.76 of the polysulfonate, polycarboxylate, or polyphosphorylated may be reacted with an acetate salt in an aqueous medium. The resulting acetic acid byproduct can then be removed leaving a polymeric salt reaction product. In exemplary embodiments, an oligodynamic metal polystyrene sulfonate is formed by concentrating PSS-H (Poly(4-styrenesulfonic acid) solution M_{w}˜75,000, 18 wt. % in H₂O, 561223 ALDRICH Sigma-Aldrich, St. Louis, Mo.) via lyophilization from 18 wt. % to 36 wt. %. Enough of the desired acetate salt is then added to obtain a quantitative loading of the desired cation. This quantitative loading can be calibrated to titer the cation concentration across a selectable range, to calibrate Cmax and Tmax of cation delivery into a wound fluid or at a wound surface, to meter antimicrobial activity to a desired level depending on wound stage, and to modify cation dosing and toxicity to balance and optimize antimicrobial activity and promotion of wound healing. The mixture is then stirred for thirty minutes at room temperature (˜20° C.) at which time a strong acetic acid smell is evident. The solution is then added dropwise to 2 L of 100% alcohol (reagent grade 90% ethanol, 5% methanol, 5% isopropanol) under high shear and stirred for 5 minutes. The precipitated polymer is filtered under vacuum and the solid washed 4× with 100% alcohol until no odor was detectable. The polymer is then collected and dried in a vacuum oven for 4-18 hours 60° C. to form the PSS-oligodynamic material. The functionalized PSS-oligodynamic material is then stored in a dry amber or opaque bottle at or below 20° C.

In another embodiment, poly(4-styrenesulfonate sodium) (PSS-Na) may be reacted to achieve calibrated loading of an antimicrobial cation, such as an oligodynamic metal. For example, PSS-Na may be solubilized in deoxygenated deionized water. To a vessel containing the PSS-Na solution, a mass of acetate salt sufficient to obtain a quantitative loading of the desired cation is added. The mixture is then stirred with a stir-plate or overhead mixer for 30 minutes at room temperature (˜20° C.) until a strong acetic acid smell is evident. The solution is then added drop wise to 2 L of 100% alcohol (reagent grade 90% ethanol, 5% methanol, 5% isopropanol) under high shear and stirred for 5 minutes. The precipitated polymer is then filtered under vacuum and the solid washed 4× with 100% alcohol and no odor is detectable. The solid is then collected and dried in a vacuum oven for 4-18 hours at 60° C. The functionalized PSS-X is stored in a dry amber or opaque bottle at or below 20° C.

In more detailed embodiments, polystyrene sulfonate (PSS) is reacted with silver acetate to form polystyrene sulfonate silver as follows:

PSS-Na+AgOAc→PSS-Ag+NaOAc or

PSS-H+AgOAc→PSS-Ag+HOAc

Polystyrene sulfonate may alternatively be reacted with zinc acetate as follows:

2(PSS-H)+Zn(OAc)₂→PSS₂-Zn+2(HOAc)

Polystyrene sulfonate may alternatively be reacted with copper acetate as follows:

2(PSS-H)+CU(OAc)₂→PSS₂-Cu+2(HOAc)

In another example, co(poly(divinyl benzene)-poly(styrene sulfonate) may be reacted with silver, copper or zinc acetate as follows:

$$co ( poly ( divinyl benzene ) \text{-} poly ( styrene sulfonate ) —H + Ag OAc \to co ( poly ( divinyl benzene ) \text{-} poly ( styrene sulfonate ) —Ag + HOAc \\ co ( poly ( divinyl benzene ) \text{-} poly ( styrene sulfonate ) —H + {Zn ( OAc )}_{2} \to {co ( poly ( divinyl benzene ) \text{-} poly ( styrene sulfonate ) )}_{2} —Zn + 2 ( HOAc ) \\ co ( poly ( divinyl benzene ) \text{-} poly ( styrene sulfonate ) —H + {Cu ( OAc )}_{2} \to {co ( poly ( divinyl benzene ) \text{-} poly ( styrene sulfonate ) )}_{2} — Cu + 2 ( HOAc )$$

In a further example, heparin or chondroitin sulfate sodium (CDS-Na), highly sulfonated glygosaminoglycans, can be solubilized in deoxygenated deionized water. To a vessel containing the heparin solution or chondroitin sulfate sodium, a mass of acetate salt sufficient to obtain a quantitative loading of the desired cation is added. The mixture is stirred with a stir-plate or overhead mixer for 30 minutes at room temperature (˜20° C.) until a strong acetic acid smell is evident. The solution is then added drop wise to 2 L of 100% alcohol (reagent grade 90% ethanol, 5% methanol, 5% isopropanol) under high shear and stirred for 5 minutes. The precipitated polymer is filtered under vacuum and the solid washed 4× with 100% alcohol until no odor is detectable. The precipitate is then collected and dried in a vacuum oven for 4-18 hours at 60° C. The functionalized heparin-X or CDS-X can be stored in a dry amber or opaque bottle at or below 20° C.

In addition to oligodynamic metals, sulfonate compounds can also be associated with one or more quaternary ammoniums. Exemplary quaternary ammonium salts include the alkyl ammonium halides such as cetyl trimethyl ammonium bromide, alkyl aryl ammonium halides such as octadecyl dimethyl benzyl ammonium bromide, N-alkyl pyridinium halides such as N-cetyl pyridinium bromide, and the like. Other suitable types of quaternary ammonium salts include those in which the molecule contains either amide, ether or ester linkages such as octyl phenoxy ethoxy ethyl dimethyl benzyl ammonium chloride. N-(laurylcocoaminoformylmethyl)-pyridinium chloride, and the like. Additional quaternary ammonium compounds include those in which the hydrophobic radical is characterized by a substituted aromatic nucleus as in the case of lauryloxyphenyltrimethyl ammonium chloride, cetylaminophenyltrimethyl ammonium methosulfate, dodecylphenyltrimethyl ammonium methosulfate, dodecylbenzyltrimethyl ammonium chloride, chlorinated dodecylbenzyltrimethyl ammonium chloride, and the like.

Quaternary ammonium compounds which are useful within the compositions and methods of the invention include those having the structural formula:

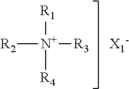

where at least one of R1, R2, R3, and R4 is a hydrophobic, aliphatic, aryl aliphatic or aliphatic aryl radical of from 6 to 26 carbon atoms, and the entire cation portion of the molecule has a molecular weight of at least 165. The hydrophobic radicals may be long-chain alkyl, long-chain alkoxy aryl, long-chain alkyl aryl, halogen-substituted long-chain alkyl aryl, long-chain alkyl phenoxy alkyl, etc. The remaining radicals on the nitrogen atoms other than the hydrophobic radicals are substituents of a hydrocarbon structure usually containing a total of no more than 12 carbon atoms. The radicals R1, R2, R3 and R4 may be straight chained or may be branched, but are preferably straight chained, and may include one or more amide or ester linkages. The radical X₁ may be any salt-forming anionic radical.

Synthetic reactions with polymers such as polysulfonates and quaternary ammoniums may be run with a complimentary cation. For example, the sulfonate group can be associated with an inorganic species such as one or more of a positively charged mono- or divalent oligodynamic metals including, but not limited to, zinc, copper, silver and bismuth. The sulfonate can also be associated with NH— or NR₅— where R₅ represents an alkyl, aryl or alkyl-aryl substituent for example. In other exemplary embodiments, the sulfonate group can be associated with one or more organic species. Suitable organic species include nitrogen containing organic species such as, an amino acid, a tetracycline, doxycycline, arginine, lysine, glutathione, lidocaine, albuterol, and/or alkyl/benzylammonium, among others. In some embodiments, the polysulfonate may be associated with numerous elemental cations and cations of compounds that have pharmacologically therapeutic value, either singularly (100% of one cation type) or in combination (two or more cations to make 100%).

In some embodiments, the cation may be differentially loaded onto the sulfonate compound. For example, the cation-sulfonate compound may contain 1%, 10%, 20%, 30% 40%, 50% or more (e.g., by dry or wet weight, or alternatively by percent substitution of available target moieties, such as Na+, as described below) of a cation such as silver. The amounts of cation such as silver attached to the sulfonate compound may be adjusted depending on the use for the compound. For example, as a wound heals, the amount of anti-microbial may be decreased slowly or adjusted depending on the bacterial load.

In exemplary embodiments using modified silver analogs (where Ag+ for Na+ substitution is carried out), a substitution of less than 100% can result in a material that retains good water solubility and has lower toxicity than the fully substituted counterpart. In alternative embodiments, the level of silver substitution can be selected in any value or range, for example 5% or 10% (5 or 10 of every Na+ substituted by Ag+), 10-20%, 20-30%, 30-50%, 50-75% or higher. This results in proportionate changes in solubility, pharmacokinetics and pharmacodynamics (including toxicity and wound healing histometrics), as described. In one working example, about 14% Ag+ substitution (i.e., −14 out of every 100 Na+ are substituted by Ag+) was utilized with discrete solubility and performance parameters observed. In another working example, about 34% Ag+ substitution was utilized, yielding distinct solubility and performance profiles. In a further example, about 79% Ag+ substitution was utilized. The amount of substitution affects several properties of interest of the derivative material. For instance, the amount of substitution prominently alters water solubility of the derivative material. In applications where enhanced water solubility of the derivative material is desired, a relatively lower percentage of Ag+ for Na+ substitution is utilized, which still affords beneficial antibacterial properties of the silver cation. Exemplary 14% and 34% substituted derivative materials were quite water-soluble, while providing strong antimicrobial properties against Staphylococcus aureus, Acinetobacter baumannii, and/or Pseudomonas aeruginosa, among others. A higher percentage of Ag+ for Na+ substitution, e.g., 79%, yields selectably lower solubility. When a polysulfonated material, for example, sodium polystyrene sulfonate (SPSS, the polysulfonated therapeutic compounds 18-sod), is 100% substituted/exchanged with a cation dissolution in deionized water is not entirely prevented.

Additional, combinatorial derivatives are provided following the same general rational design scheme. For example, with the antibiotic mafenide, a derivatized material is provided by combining an equimolar amount of NaPSS in deionized water to yield NaPSS-maf, where mafenide cations and sodium cations each occupy approximately equal numbers of sulfonate group sites in the polymer (isolated by lyophilizing the solution to yield a solid mixed salt). Such a mixed complex can be further elaborated into a multi-active complex by adding silver substitution, or other cation substitution, into the combinatorial matrix—across the full spectrum of species disclosed herein to yield additional novel derivative therapeutics.

Na/Ag (mixed sodium cation/silver cation) at various levels of silver cation substitution (14, 34, and 78 mol %) are effective to kill a diverse array of bacterial organisms. The higher the level of silver substitution, the lower the dosage, concentration and duration of exposure is required for the polysulfonated therapeutic compounds to kill bacterial target pathogens. Exemplifying the facility of this calibration (e.g., of silver loading versus antimicrobial efficacy), polysulfonated therapeutic compounds are effective in a micromolar range for 14% Ag substitution, in a nanomolar range for 34% substitution, and in a subnanomolar range for 78% substitution. However, as the silver cation substitution increases the cytotoxicity to neonatal fibroblasts increases as well, so loading will be optimally calibrated with wound status (e.g., nature and extent of wound, extent of microbial colonization, wound stage and histology, etc.)

The compounds and compositions of the invention may additionally be modified by selective cross-linking to alter solubility and pharmacokinetic/pharmacodynamics properties of the compositions. Just as the cation-exchange modifications to the polysulfonated therapeutic compounds need not be all (100%), cross-linking substitutions introduced to alter the solubility of the polysulfonated therapeutic compounds as described above need not be all (100%) or nothing (0%) and may be engineered to any desired range in between. Certain embodiments employ partial modifications to achieve useful resultant or derivative materials that exhibit metered dosing or release kinetics of antimicrobial agents. Partial cross-linking substitution also leaves sulfonate groups available for cation binding and can produce derivative materials that incorporate multiple active agents (e.g., silver and a cationic antibiotic or other cationic secondary therapeutic agent, such as an anti-inflammatory or local anesthetic), or which have other characteristics different from those of the unmodified polysulfonate and fully substituted derivative materials. To modify these characteristics and achieve projected metered changes in pharmacology and therapeutic benefit, cross-linking substitution will routinely be selected among optional values and ranges of between 5-10% (of available cross linkable sites/partners), 10-20%, 25-40%, 40-60%, 60-90% or higher.

Each of the antimicrobial compounds, compositions, formulations, methods and devices for treating wounds will exhibit profound clinical advantages in terms of antimicrobial efficacy and wound healing promotion activity, and in various embodiments and combinations described here will further provid novele antiproteolytic and macromolecule protecting activities (i.e., preventing destruction and/or deactivation, and/or elevating levels of, beneficial endogenous cytokines, growth factos, extracellular matrix components, glutathione and glutathione transferase, among other wound healing promoting biomolecules). In certain embodiments, the compositions and methods of the invention will exhibit antimicrobial activity that is substantially greater than that exhibited by first line, existing antimicrobial topicals and dressings, for example silver sulfadiazine (SSD). The superior antimicrobial performance of the invention will range in distinct aspects from at least 50% increased antimicrobial activity compared to SSD, to two-fold, five-fold, ten-fold, 25-fold, 50-fold, 100-fold or even higher comparative activity against any one or more of the microbial pathogens identified herein. This efficacy can be demonstrated by any number of assays and clinical tests, most simply by determining bacterial load according to standard assays employing side-by-side infected wound models, pre- and post-treatment.

Comparable benefits are provided by the invention in terms of enhanced wound healing (while maintaining potent antimicrobial activity)—marked by reduced toxicity to cells involved in wound healing processes (e.g., epithelial cells, endothelial cells, fibroblasts, hematopoietic cells, leukocytes, lymphocytes, neutrophils and granulocytes). Both in vitro and in histometric assays in vivo, toxicity exhibited by the compositions and methods of the invention (attending a fixed level of antimicrobial activity) will be at least five-10% reduced, 20-30% reduced, 30-50% reduced, 50-75% reduced, up to 80-90%, 95%, or even 98% reduced, compared to toxicity determined for SSD at the same level of antimicrobial activity (for any one or more cell type(s) or histometric indices identified herein). In exemplary embodiments, each individual and combinatorial formulation and method of the invention will display wound healing promoting activities superior to SSD and other topical antimicrobial wound treatments—as determined by conventional histometric or other clinical observations (e.g., identification/quantification of erythema, macular/papular eruption, ulcerating dermatitis, vascular degeneration, gross anatomic and histological markers of wound healing status (e.g., re-epithelization, status of granulation tissue determined by staining for Von Willebrand factor, contraction), symptoms of sepsis, blood markers of organ dysfunction, general illness, etc.)

In related embodiments the invention provides additional clinical advantages by reducing inflammation in antimicrobial treated wounds. As compared to SSD and other topical antimicrobial treated wounds, the compositions and methods of the invention will exhibit at least 10-20% reduced inflammation (e.g., measuring myeloperoxidase (MPO) activity, a standard marker for neutrophil activity in wound exudates or biopsies), up to 25-40%, 40-60%, 60-90% or higher.

For each measure of efficacy described herein, combinatorial compounds, formulations and methods combining more than one aspect of the invention (e.g., an oligodynamic metal, a thiol, a polysulfonate, differentially loaded or cross-linked derivative compounds, secondary therapeutic agents like cationic antibiotics, anesthetics, growth factors, etc.) will exhibit substantial improved properties (antimicrobial, antiproteinase, macromolecule protective, anti-inflammatory, wound healing promoting, etc.) compared to either aspect alone (either drug agent, or drug agent and adjunctive composition, modification or method), or compared to different, multiple agents or methods lacking the one or more combinatorial aspect subjected to comparison. Combinatorial formulations and coordinate treatment protocols employing multiple aspects or embodiments of the invention need not be synergistic to evince surprising combinatorial efficacy, rather the discovery of combinatorial embodiments may simply yield surprising additive benefits in terms of enhanced clinical efficacy in one measure, or a broadened array of activities yielding combinatorially distinct clinical benefits.

In additional aspects of the invention, polysulfonated therapeutic compounds as described herein can be formulated as a salt with three or more cations. For example, the polysulfonated therapeutic compounds may include partial antibiotic incorporation (such as mafenide), partial for Na+ substitution, and partial Ag+ substitution to produce a material that has a combination of Ag+, Na+, and mafenide cation incorporated. It is also possible to achieve this combination (cation) salt by combining appropriate amounts of a sodium salt of a polysulfonated therapeutic compounds, a mafenide salt of a polysulfonated therapeutic compounds, and a 100% silver salt or mixed sodium-silver salt of a the polysulfonated therapeutic compounds, dissolving all three into de-ionized water to achieve a solution and subsequently lyophilizing the solution to a solid. In solution, the salts which are all substituted at 100% of their respective cations, exchange cations rapidly reaching a mixed cation polysulfonated material.

In certain embodiments, sulfonated cations such as PSS-Ag may further be attached to thiol compounds. For example, PSS-Ag may be combined with glutathione according to the following reaction scheme:

PSS-Ag+GSH→PSS-Ag-GSH

The addition of the thiol compounds to sulfonated cations increases solubility of the cation, increases the stability of the formulation, increases the biocompatibility of the formulation and additionally increases the activity of the cation against some organisms such as P. aeruginosa.

In one of many working examples demonstrated here, a petrolatum-based (oil-in-water) emulsion (ointment/cream) formulation using a PSS-Ag active agent was prepared by blending the appropriate amount of active agent and glutathione (GSH) into the ointment base. To test the efficacy of these formulations, the ointments/creams were plated onto lawns of bacteria including: Staphylococcus aureus, Pseudomonas aeruginosa, and Enterococcus faecalis.

The data in Table 1 summarizes the results of Kirby-Bauer antimicrobial effectiveness test data for formulations with and without varying concentrations of reduced glutathione (GSH) for cream and gel topical formulations. Values are averaged for results versus Pseudomonas aeruginosa, Acinetobacter baumannii, Klebsiella Pneumoniae, and MRSA. Caffeine silver carbene (CSC) is a methylated caffeine carbene silver compound stabilized with acetic acid, and pyrimidine silver carbene (PSC) is a dichloro-, methyl-, propanol-substituted pyrimidine carbene silver compound stabilized with acetic acid. Additional carbenes known to those of skill in the art may also be used.

TABLE 1
Average zone radius of bacterial growth in lawns of bacteria onto which
was plated various forms of silver and GSH

| | | | average |
|---|---|---|---|
| | | | Zone |
| | Silver | [GSH] | radius |
| Vehicle | compound | % (w/w) | (cm) |
| Gel | silver | 3 | 0.06 |
| | sulfadiazine | 2 | 0.19 |
| | | 1 | 0.32 |
| | | 0.5 | 0.46 |
| | | 0 | 0.25 |
| | Polystyrene | 4 | 0.21 |
| | sulfonate | 3 | 0.53 |
| | silver | 2 | 0.69 |
| | | 1 | 0.45 |
| | | 0 | 0.21 |
| | caffeine | 4 | 0.00 |
| | silver | 3 | 0.00 |
| | carbene | 2 | 0.60 |
| | | 1 | 0.55 |
| | | 0 | 0.14 |
| | pyrimidine | 4 | 0.48 |
| | silver | 3 | 0.63 |
| | carbene | 2 | 0.53 |
| | | 1 | 0.50 |
| | | 0 | 0.15 |
| Cream | silver | 4 | 0.10 |
| | sulfadiazine | 3 | 0.18 |
| | | 2 | 0.32 |
| | | 1 | 0.44 |
| | | 0 | 0.19 |
| | caffeine | 4 | 0.00 |
| | silver | 3 | 0.00 |
| | carbene | 2 | 0.45 |
| | | 1 | 0.54 |
| | | 0 | 0.15 |
| | Polystyrene | 4 | 0.31 |
| | sulfonate | 3 | 0.60 |
| | silver | 2 | 0.59 |
| | | 1 | 0.52 |
| | | 0 | 0.19 |

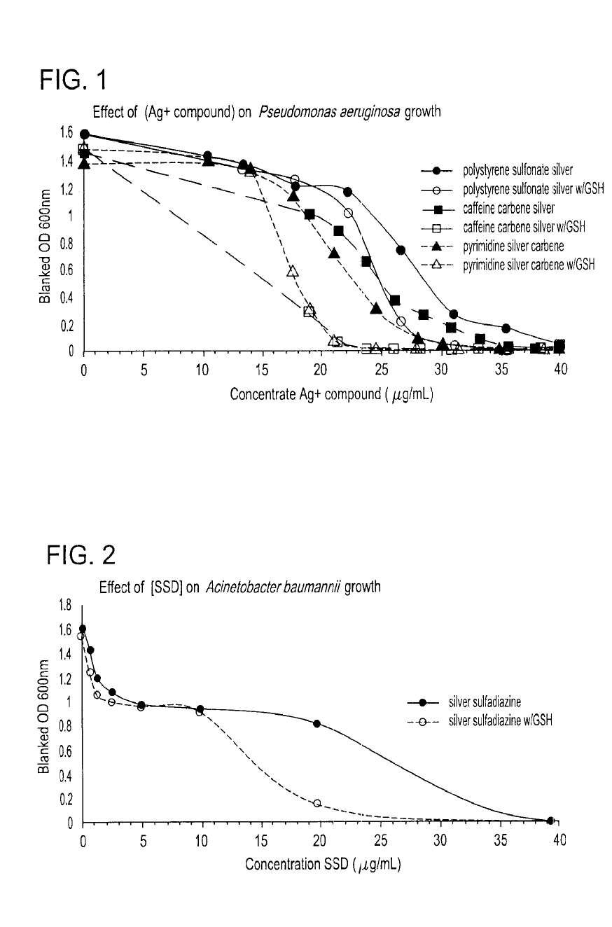

The addition of GSH to the oligodynamic metal compositions was shown to increase effectiveness (ability to kill bacteria) by a factor of 3 to 4. As shown in FIGS. 1, 2, and 3, silver and glutathione compounds were effective in decreasing the amounts of Pseudomonas aeruginosa, Acinetobacter baumannii, and Klebsiella Pneumoniae alone.

Additionally, as shown in Table 2, combinations of various forms of silver and different amounts cysteine similarly plated against lawns of Pseudomonas aeruginosa, Acinetobacter baumannii, Klebsiella Pneumoniae, and MRSA were also effective in decreasing bacterial counts. Zone radii are averaged for Pseudomonas aeruginosa, Acinetobacter baumannii, Klebsiella Pneumoniae, and MRSA.

TABLE 2
Average zone radius of bacterial growth in lawns of bacteria onto which
was plated various forms of silver and cysteine

| | | | | average |
|---|---|---|---|---|
| | | | | Zone |
| | Silver | [compound] | [cysteine] | radius |
| Vehicle | compound | % (w/w) | % (w/w) | (cm) |
| Cream | caffeine silver | 3.2 | 1.5 | 0.38 |
| | carbene | | 2 | 0.03 |
| | | | 2.25 | 0.01 |
| | | | 2.5 | 0.00 |
| | Pyrimidine | 5.1 | 3.25 | 0.05 |
| | silver | | 3.5 | 0.01 |
| | carbene | | 3.75 | 0.04 |
| | | | 4 | 0.00 |
| Gel | caffeine silver | 3.2 | 1.5 | 0.28 |
| | carbene | | 2 | 0.07 |
| | | | 2.25 | 0.04 |
| | | | 2.5 | 0.00 |
| | Pyrimidine | 5.1 | 3.25 | 0.03 |
| | silver | | 3.5 | 0.03 |
| | carbene | | 3.75 | 0.02 |
| | | | 4 | 0.00 |

The addition of cysteine to the silver carbene compound formulation was shown to improve the zone of inhibition by a factor of about 2.7 in the cream and by a factor of about 2.2 in the gel in comparison to silver carbene alone.

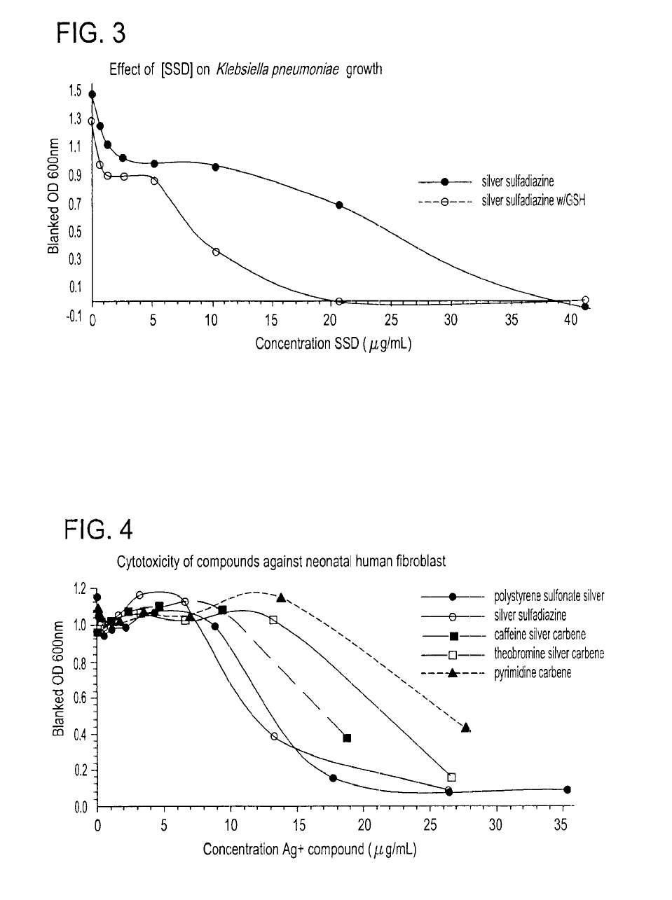

Referring to FIGS. 1, 2, and 3, the plots demonstrate the relationship between concentrations of silver compounds versus OD600 nm, which correlates with bacterial density. The specific effectiveness of Ag⁺ compounds on Pseudomonas aeruginosa is shown in FIG. 1, Acinetobacter baumannii in FIG. 2, and Klebsiella Pneumoniae in FIG. 3.

TABLE 3
Cytotoxicity for multiple silver compounds with and
without glutathione on human neonatal fibroblasts.

| | | predicted factor |
|---|---|---|
| | IC50 | of increased |
| | [μg | tolerance/ |
| | compound/ | decreased |
| Compound | mL media] | cytotoxicity |
| styrene sulfonate silver | 14 | |
| styrene sulfonate silver w/GSH | 55-96 | 4 to 7 |
| silver sulfadiazine | 12 | |
| silver sulfadiazine w/GSH | 46-57 | 4 to 5 |
| caffeine silver carbene | 17 | |
| caffeine silver carbene w/GSH | 66-149 | 4 to 9 |
| theobromine silver carbene | 21 | |
| theobromine silver carbene w/GSH | 85-128 | 4 to 6 |
| pyrimidine carbene | 25 | |
| pyrimidine carbene w/GSH | 100-125 | 4 to 5 |

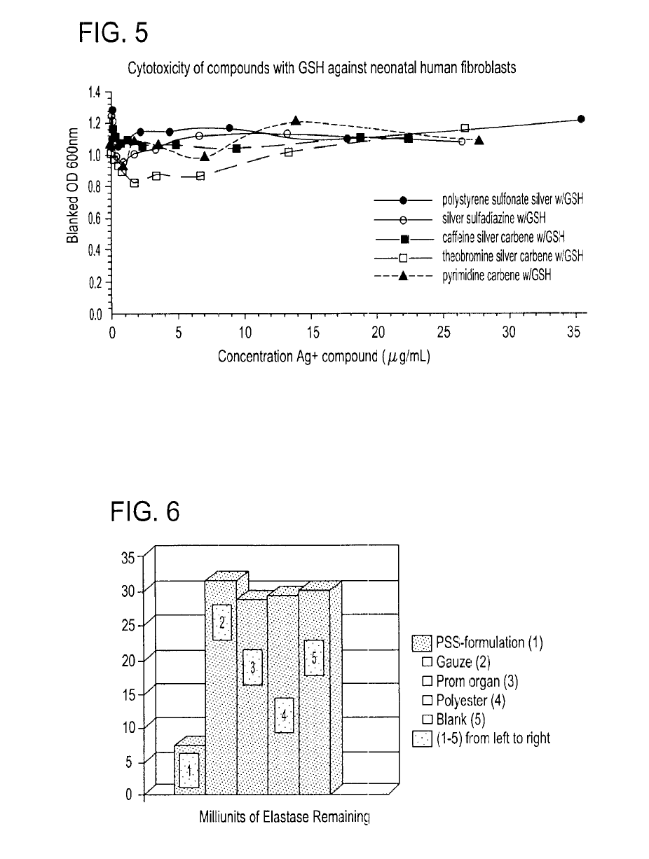

Referring to Table 3, the tabulated data summarizes cytotoxicity data (via MTS assays) for multiple silver containing compounds with and without the addition of reduced glutathione. The IC₅₀ value indicates the concentration at which the target compound inhibits 50% (in this case, kills) of human neonatal fibroblasts. Referring to FIGS. 4 and 5, the graphs demonstrate the results of the MTS cell viability assay on human neonatal fibroblasts. The plots demonstrate the relationships of concentration of silver compounds versus OD490 nm, the absorption band maximum for MTS. Higher absorption values indicate there are more viable, metabolizing cells present. Lower values indicate that there are fewer viable, metabolizing cells present. Therefore, the absorption at 490 nm correlates to the number of viable cells, which can be used to interpret the cytotoxicity of the compound the cells were exposed to.

The effectiveness of varying amounts of glutathione (GSH) in different formulations (cream vs. gel) was evaluated using a Kirby-Bauer assay. Table 4 shows the effectiveness of the various silver sulfadiazine/glutathione (SSD/GSH) ratios against Pseudomonas aeruginosa in gel/cream formulations.

TABLE 4
SSD zone of inhibition

| Wt % | SSD Cream (Avg. Zone | SSD Gel (Avg. Zone |
|---|---|---|
| GSH | radium mm) | radius mm) |
| 0 | 2.5 | 1.9 |
| 0.5 | N/A | 4.6 |
| 1 | 4.4 | 3.2 |
| 2 | 3.2 | 1.9 |
| 3 | 1.8 | 0.6 |
| 4 | 1 | N/A |

Similar results were obtained for multidrug resistant forms of Acinetobacter baumannii-calcoaceticus, Klebsiella pneumonia, and MRSA.

The addition of GSH additionally decreased the toxicity of SSD and GSH in situ as shown in FIGS. 4 and 5. Unexpectedly, in samples subjected to accelerated aging (based on tetrazolium reduction), there was a ten-fold increase in the IC₅₀ for dermal fibroblasts and a half-log decrease in the MIC₉₀ for both Pseudomonoas aeruginosa and MRSA as shown in Table 5.

TABLE 5
Week 12 Accelerated Aging Samples Evaluations (approximate MIC₉₀ and IC₅₀)

| | MIC₉₀ | | Therapeutic | MIC₉₀ | | Therapeutic |
|---|---|---|---|---|---|---|
| Antimicrobial | Pseudomonas | | index BI = | staphylococcus | | index (BI = |
| Agent | aeruginosa | 10k IC₅₀ | IC₅₀/MIC₉₀) | aureus | 10K IC₅₀ | IC₅₀/MIC₉₀) |
| Sulfamylon | 1400 | 50 | 0.04 | 1400 | 50 | 0304 |
| Cream | | | | | | |
| Thermazene | 96 | 15 | 0.16 | 96 | 15 | 0.16 |
| SSD Cream | | | | | | |
| SSD:GSH | 35 | 156 | 4.4 | 35 | 156 | 4.4 |
| Gel | | | | | | |

The lower toxicity coupled with equivalent or improved MICs against relevant pathogenic bacteria permits safer treatment over a wider therapeutic window.

Biocompatibility (BI) indexes greater than 1.0 (MIC₉₀s<IC₅₀s) indicate that a compound can be effective against bacteria with minimal or reduced cytotoxicity (24). In a pilot accelerated aging study at week 16, the BI of SSD cream without GSH used as a control (Thermazene®, Ascend Laboratories, LLC) was 0.16 ([15 μg/mL]/[96 μg/mL]) reflecting high cytotoxicity. At t₀ the Ascend (Thermazene®) cream provided a BI of 0.60 ([15 μg/mL]/[25 μg/mL]) demonstrating that the drug becomes less effective against bacteria (increased MIC) over time while maintaining the same overall cellular toxicity by MTS assay. In a gel formulation of a therapeutic compound at week 16, the addition of GSH to SSD reduced the in vitro cytotoxicity (increased IC₅₀) and improved the antimicrobial effectiveness (decreased MIC₉₀) resulting in a BI of 4.40 ([156 μg/mL]/[35 μg/mL]) representing a 28× improvement versus Thermazene®. Although some of the demonstrated difference in formulation toxicity (SSD cream vs. SSD:GSH gel) can be attributed to the vehicle, i.e. cream vs. gel with the petroleum-based cream demonstrating slightly higher cellular toxicity, this is negligible as we have confirmed that SSD alone is significantly more cytotoxic (via MTS assay) than SSD:GSH complex

At t₀ the BI of the gel formulation is about 3.0 which is currently believed to be due to the slow, inefficient formation of the SSD:GSH complex in situ. Pre-reaction of SSD with GSH yields a yellow complex from two white starting materials which is more efficient and more consistent than in situ SSD:GSH generation.

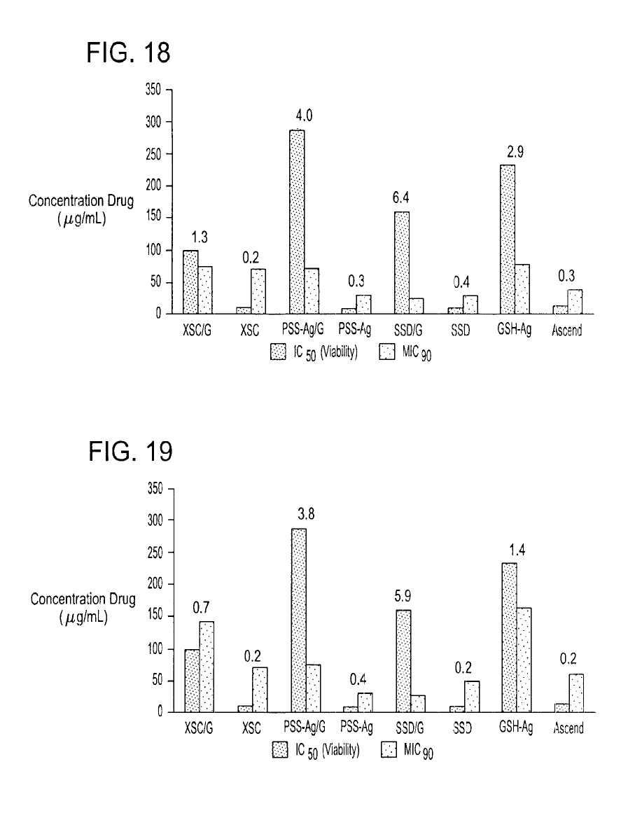

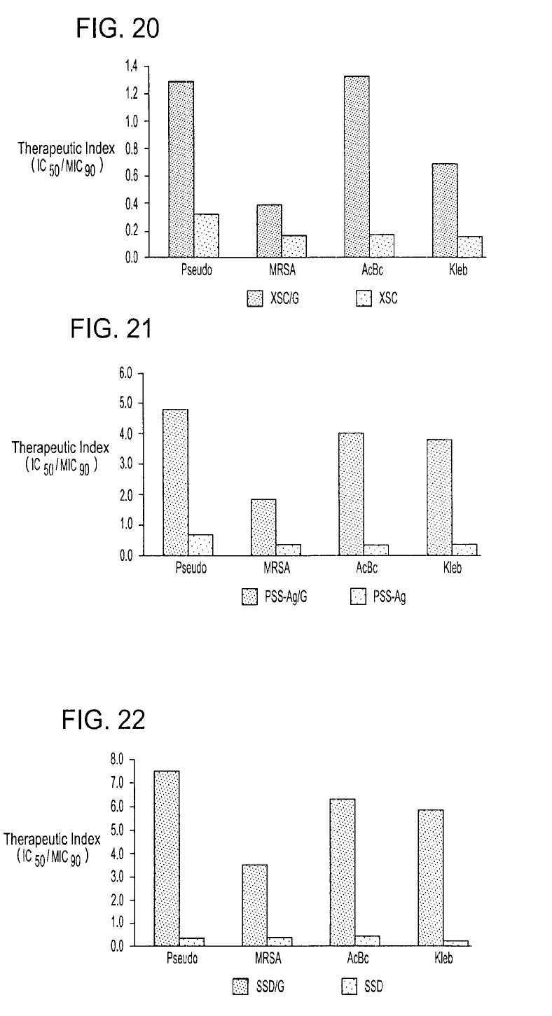

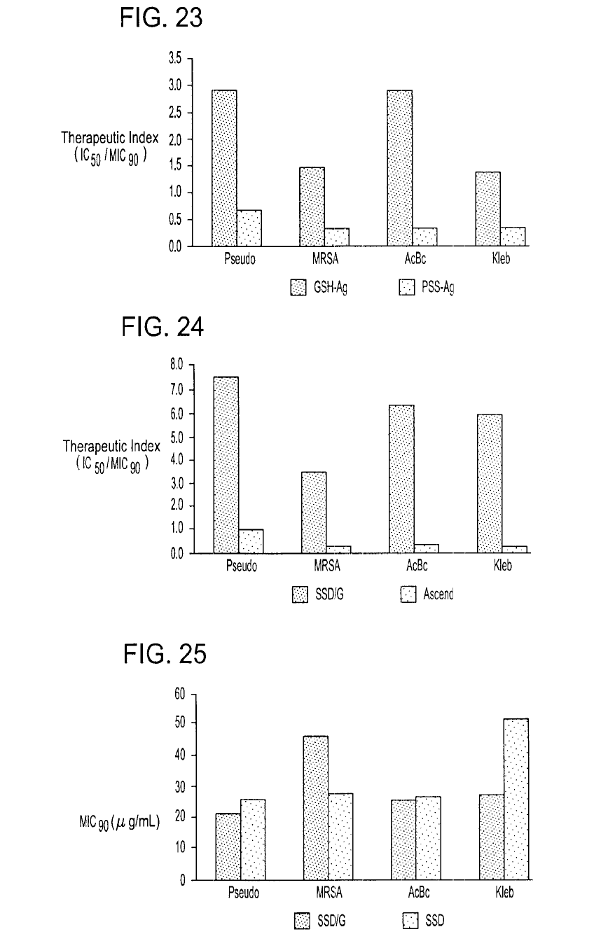

As shown in FIGS. 16, 17, 18 and 19, the addition of glutathione to silver compounds increases the therapeutic index (IC₅₀/MIC₉₀) of formulations of xanthine-based silver carbene (XSC), poly(4-styrenesulfonate) silver (PSS-Ag), silver sulfadiazine (SSD), glutathione silver (GSH-Ag) and 1% silver sulfadiazine in a white water dispersible cream (Ascend® Laboratories, Montvale, N.J.) against Pseudomonas aeruginosa, Acinetobacter baumannii-calcoaceticus, Klebsiella pneumonia, and MRSA. Specific comparisons of each compound with and without GSH are shown in FIGS. 20-25.

Accelerated aging studies indicate that the SSD:GSH complex is more stable than SSD alone. As demonstrated in FIGS. 29-31, thiol and sulfide containing formulations such as silver sulfadiazine GSH, polystyrene sulfonate silver GSH gel and xanthine based silver carbene GSH gel retain their effectiveness against Pseudomonas aeruginosa and MRSA while also maintaining their biocompatibility (FIG. 29).

Severe trauma leads to up regulation of free radical and reactive oxygen species, impairing antioxidant defense mechanisms and rendering burn victims more susceptible to oxygen free radical mediated injury. Glutathione is an antioxidant. Therefore glutathione activity was evaluated in a freshly prepared gel formulation (1 wt % SSD/0.5 wt % GSH), and a 16-week aging sample (40° C.) using a Promega GSH-Glo assay (Madison, Wis.). The study determined that the glutathione present in the gel formulation is in its reduced form (minimal GSH oxidation) and that the formulations likely retain their antioxidant property, with a slight reduction (˜10%) observed in the formulation following 16 weeks at 40° C. (to simulate 64 weeks @ room temperature). The SSD:GSH metal-ligand complex therefore does not inhibit GST, a known silver binding protein that plays an important role in detoxification and is implicated in binding Ag⁺ and staining wounds treated with silver.

The addition of an oligodynamic metal-thiol compound has many attractive properties including increasing the solubility of the metal cation. Using Ag-Fix test strips (Macherey-Nagel, Inc., Bethlehem, Pa.) it was determined that for the complex at 0.5 and 1.0 wt % GSH, silver ion solubility increases by at least three logs versus silver sulfadiazine alone. Additionally, the silver carbene compound gels in particular can be made to turn clear/transparent with the addition of the appropriate amount of GSH with concentrations of the silver carbene compounds in the gels over a large range of concentrations from about 0.1 wt % to 25 wt % or greater, increasing their cosmetic appeal.

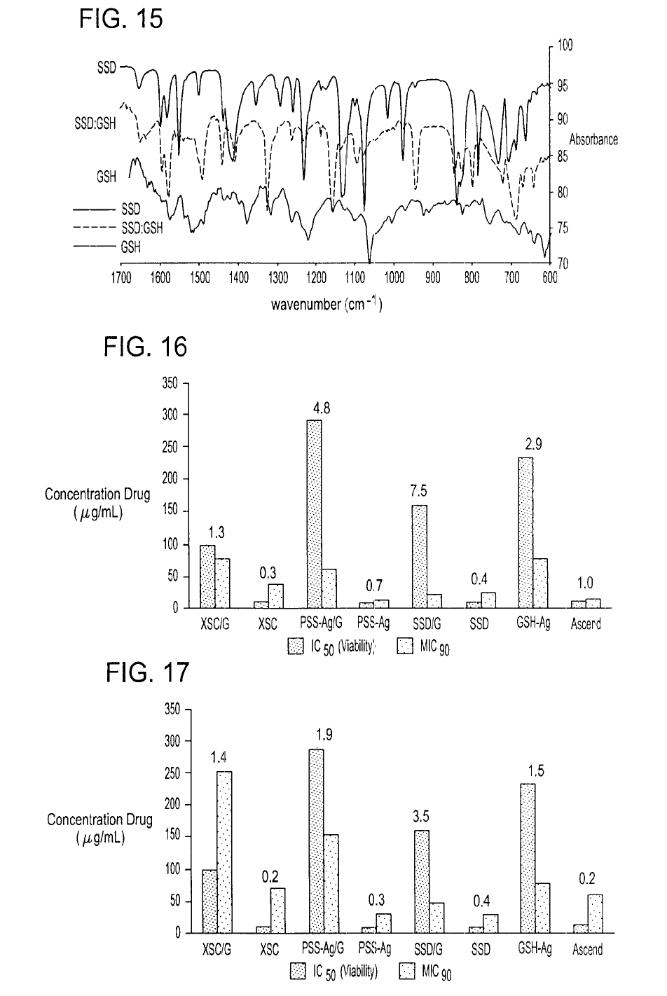

The addition of glutathione to silver sulfadiazine cream creates an SSD:GSH complex that is distinct from either SSD or GSH alone as shown in FIG. 15. This spectrum is consistent with a new compound vs. a mixture as evident by new infrared (IR) peaks. Furthermore, the disappearance of the sulfhydryl stretch (not shown at 2550 cm⁻¹) strongly suggests the formation of a structurally-complex silver thiolate. SSD and GSH react to form what appears to be a homogeneous, structurally complex metal-ligand compound (as opposed to a mixture of compounds) with a unique IR spectrum, altered solubility, different color, and an IC₅₀ six to ten times greater (in a gel) than Thermazene® cream alone while possessing a decreased MIC₉₀ against a range of multidrug-resistant clinical isolates.

The therapeutic compositions described herein may be formulated for topical application or attached to a solid support. In some embodiments, the topical carrier used to deliver the compound is an emulsion, gel or ointment. In other embodiments, the therapeutic compounds as described herein may be formulated in a spray formulation.

Emulsions, such as creams and lotions are a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions, such as creams and lotions that can be used as topical carriers and their preparation are disclosed in Remington: The Science And Practice Of pharmacy 282-291 (Alfonso R. Gennaro ed. 19th ed. 1995), incorporated herein by reference.

In some embodiments, the sulfonated compounds and the thiol compounds may be mixed in separate forms into a mixture, allowing the association of the sulfonated compounds and the thiol compounds to take place within the formulation. For example the sulfonated compounds and the thiol compounds may be mixed in a liquid or semisolid mixture such as an ointment comprising petrolatum, fatty alcohol (stearyl), emollient (isopropyl myristate), emulsifying agent (polyoxy(40) stearate, sorbital monooleate), humectant (propylene glycol), and sterile deionized water, among others.

Ointments may be homogeneous, viscous, semi-solid preparation, most commonly a greasy, thick oil (oil 80%-water 20%) with a high viscosity. The ointment can be used as an emollient or for the application of active ingredients to the skin for protective, therapeutic, or prophylactic purposes where a degree of occlusion is desired.

A cream is an emulsion of oil and water in approximately equal proportions. It penetrates the stratum corneum outer layer of skin well. Cream is generally thinner than ointment, and maintains its shape when removed from its container. It tends to have moderate moisturizing activity.

The vehicle of an ointment/cream is known as the ointment base. The choice of a base depends upon the clinical indication for the ointment. The different types of ointment bases include, but are not limited to: hydrocarbon bases, e.g. hard paraffin, soft paraffin, microcrystalline wax and ceresine; absorption bases, e.g. wool fat, beeswax; Water soluble bases, e.g., macrogols 200, 300, and 400; Emulsifying bases, e.g. emulsifying wax, Vegetable oils, e.g. olive oil, coconut oil, sesame oil, almond oil and peanut oil. The therapeutic compounds are dispersed in the base and later get divided after the drug penetrates into the wound. Ointments/creams can be formulated incorporating hydrophobic, hydrophilic, or water-emulsifying bases to provide preparations that are immiscible, miscible, or emulsifiable with skin secretions. They can also be derived from fatty hydrocarbon, absorption, water-removable, or water-soluble bases.

For example, a cream/ointment base can contain the active agent, white petrolatum, water, allantoin, EDTA, Stearyl alcohol, Brij 721, Brij 72, methylcelluloses, isopropyl myristate, Sorbitan monooleate, Polyoxyl 40 stearate, butylated hydroxytoluene, propylene glycol, methylparaben, propylparaben, deionized water to 100%, buffer to neutral pH. In some embodiments, the active ingredient may combine or be combined with components for the cream/ointment base, for example an ethylenediaminetetraacetic acid (EDTA)-silver salt:GSH complex may form.

In another embodiment, the topical carrier used to deliver a compound of the invention is a gel, for example, a two-phase gel or a single-phase gel. Gels are semisolid systems comprising suspensions of small inorganic particles or large organic molecules interpenetrated by a liquid. When the gel mass comprises a network of small discrete inorganic particles, it is classified as a two-phase gel. In some embodiments the liquid may be water or another aqueous media and the gel mass is defined as a hydrogel. Hydrogels can include, but are not limited to, alginates, polyacrylates, polyalkylene oxides, and/or poly N-vinyl pyrrolidone). The hydrogel may also be amorphous, i.e. a viscous gel as opposed to a solid such as a formulation of carboxymethylcellulose containing a humectant such as propylene glycol or glycerin. Exemplary amorphous hydrogels include, but are not limited to, maltodextra-beta glucan, acemannan, carboxymethylcellulose, pectin, xanthan gum, collagen, keratin and honey.

Single-phase gels consist of organic macromolecules distributed uniformly throughout a liquid such that no apparent boundaries exist between the dispersed macromolecules and the liquid. In some embodiments, the therapeutic compounds may be in a solid form resin that turns to liquid when hydrated with an ionic solution. Suitable gels for use in the invention are disclosed in Remington: The Science And Practice Of Pharmacy 1517-1518 (Alfonso R. Gennaro) ed. 19th ed. 1995), hereby incorporated herein by reference. Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002), each incorporated herein by reference.

Polymer thickeners (gelling agents) that may be used in formulations described herein include those known to one skilled in the art, such as hydrophilic and hydroalcoholic gelling agents frequently used in the cosmetic and pharmaceutical industries. In some embodiments, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably the gelling agent comprises between about 0.2% to about 4% by weight of the composition. More particularly, the preferred compositional weight percent range for “CARBOPOL®” is between about 0.5% to about 2%, while the preferred weight percent range for “NATROLSOL®” and “KLUCEL®” is between about 0.5% to about 4%. The preferred compositional weight percent range for both “HYPAN®” and “STABILEZE®” is between 0.5% to about 4%.

“CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

For example, the gel base can contain the active agent, hydroxyethyl cellulose, hydroxypropyl cellulose, carboxymethyl cellulose sodium, propylene glycol, EDTA, glycerin, methyl paraben, propyl paraben, DI water to 100%, buffer to neutral pH.

In some embodiments, the compounds described herein may be covalently cross-linked with a diamine and a coupling agent. This may form a sulfonamide at some or all junctions where the diamine links to the sulfonic acid groups of some of the compounds described herein. In some cases, the cross-linking of the polysulfonated material can serve to alter the solubility of the polysulfonated material. The solubility is dependent upon the number of cross-link points introduced. Examples of cross-linkers can include peptides, aromatic or aliphatic diamines, diaminosaccharide and the like. Coupling agents can include 2-(1H-7-Azabenzotriazol-1-yl)-1,1,3,3-tetramethyl uranium hexafluoro phosphate (HATU), or 0-Benzotriazole-N,N,N′,N′-tetramethyl-uronium hexafluoro-phosphate (HBTU), for example but may include pretreatment of the acid form of the polymer with thionyl chloride in order to yield the sulfonyl chloride which will react with an amine to form a sulfonamide. Furthermore, in cases where the cross-linker is a peptide sequence, one or more of the peptide bonds within the peptide may be designed to be susceptible to proteolytic cleavage.

The hydrogel can be cross-linked in the presence of, and/or blended with, the therapeutic compound to form a solid blend. The hydrogel can be polyethylene glycol-based and/or polyvinyl alcohol-based, for example. In accordance with other embodiments of the disclosure, the therapeutic compounds such as polysulfonated therapeutic compounds can be dispersed into a solid matrix of cross-linked acrylic acid-based polymer such as methacrylic acid or any of its esters including poly(2-hydroxy ethyl methacrylate) (HEMA), polypropylene oxide, polyethylene oxide, polyvinyl alcohol, a polyurethane, a polyester, alginate, silicone, hydrocolloid, and/or other hydrogels, or an alkylene polymer (polyalkylene) such as polypropylene or polyethylene. Further, individual particles can include poly(N-vinyl pyrrolidone), poly(vinyl alcohol), poly(acrylic acid), polyacrylamide including poly(N-isopropylacrylamide), poly(ethylene-co-vinyl acetate), poly(ethylene glycol)/polyethylene oxide, poly(methacrylic acid), polyurethanes, and silicones, among others.

In accordance with another implementation, therapeutic compounds as described herein can be coated on or mixed with a biodegradable polymer. Exemplary biodegradable polymers include, but are not limited to, lactide/glycolides, polyglycolides, polyorthoesters, and/or polylactides, polycaprolactones, polydioxanones, starches, cellulose, chitosan, and cross-linked natural polymers such as collagen, gelatin or elastin.

In other embodiments, the topical carrier is an aqueous solution or suspension. An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers.

An aqueous suspension or solution/suspension of the present invention is preferably viscous or muco-adhesive, or even more preferably, both viscous and muco-adhesive. Additional suitable aqueous formulations are disclosed in Remington: The Science and Practice Of Pharmacy 1563-1576 (Alfonso R. Gennaro ed. 19th ed. 1995), incorporated herein by reference. Other suitable aqueous topical carrier systems are disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); U.S. Pat. No. 6,465,464 (issued Oct. 15, 2002), all incorporated herein by reference.

The topical formulations as described herein may further include an excipient including, but not limited to, protectives, adsorbents, demulcents, emollients, preservatives, antioxidants, moisturizers, buffering agents, solubilizing agents, skin-penetration agents, and surfactants.

Suitable protectives and adsorbents include, but are not limited to, dusting powders, zinc stearate, collodion, dimethicone, silicones, zinc carbonate, aloe vera gel and other aloe products, vitamin E oil, allatoin, glycerin, petrolatum, and zinc oxide.

Suitable demulcents include, but are not limited to, benzoin, hydroxypropyl cellulose, hydroxypropyl methylcellulose, and polyvinyl alcohol.

Suitable emollients include, but are not limited to, animal and vegetable fats and oils, myristyl alcohol, alum, and aluminum acetate.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

Suitable antioxidants include, but are not limited to, ascorbic acid and its esters, sodium bisulfite, butylated hydroxytoluene, butylated hydroxyanisole, tocopherols, and chelating agents like EDTA and citric acid.

Suitable moisturizers include, but are not limited to, glycerin, sorbitol, polyethylene glycols, urea, and propylene glycol.

Suitable buffering agents for use with the invention include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates.

The compounds described herein can additionally be blended with naturally occurring polymers that include chitosan, hyaluronic acid, and starch, among others.

In some embodiments, the compounds described herein may be formulated to be insoluble in deionized water, but soluble in ionic solutions with solubility mediated by the ionic strength. Thus dissolution of the salt and subsequent release of each of the two or more species present in the salt can be modulated by the ionic concentrations, for example, found in biological fluids. Such an occurrence can allow sulfonated compounds as described herein to be prepared as an insoluble salt in deionized water where two aqueous solutions (for example one of sodium polystyrene sulfonate (SPSS) and one of doxycycline hydrochloride) are combined to yield a deionized water-insoluble polystyrene sulfonate salt (for example doxycycline (PSS-Dox)) which forms as a precipitate that settles out following the combination of the two deionized water solutions. The sulfonated compounds as described herein, in some cases, can be obtained in a relatively pure form by simple filtration. The deionized water-insoluble salt can be slowly dissolved into an isotonic aqueous saline solution, or biologically equivalent solution with sodium, potassium, and/or calcium ions where these ions can exchange with the exemplary doxycycline ions in order to yield SPSS (or the calcium or potassium salt of PSS) and doxycycline hydrochloride. As used herein, aqueous solutions containing sodium, potassium, and/or calcium ions (simple and relevant biologically relevant salts), as well as amino acids, proteins; peptides or the like are referred to as “aqueous media”. Such solutions may include but are not limited to phosphate buffered saline solution (PBS), saline, serum (including fetal bovine serum and human serum), and other biological media. Thus, these deionized water salts can be effective “controlled-release” compounds when placed into or onto biological systems where biological fluids provide the exchange medium for dissolution. The timescale of dissolution is driven by the cation concentration and the rate of cation exchange. Salts that are insoluble in deionized water can interact with the patient's bodily fluids which facilitate the slow dissolution (and subsequent ionization) of the polysulfonated therapeutic compounds by cation exchange thus leading to protease inhibition and bacterial organism control. In other cases, the polysulfonated therapeutic compounds can remain completely water-soluble even when 100% exchange of the replacement cation is carried out. For example, a combination of sodium polystyrene sulfonate (SPSS) and mafenide acetate (4-(aminomethyl)benzenesulfonamide acetate salt) in a 1:1 molar combination results in a clear solution that when lyophilized yields a white powder mixture consisting of mafenide polystyrene sulfonate and sodium acetate.

In other embodiments, the therapeutic agents may be formulated as a hydrogel or film forming agent to form a flexible film over the wound after the application of the formulation including the therapeutic agent. In one embodiment, the film forming agent is flexible collodion. In another embodiment, the film forming agent is miscible with the skin permeation agent and the organic solvent. The film forming agent forms a moisture-resistant film that adheres to the infected site after application, forming a non-occlusive membrane which allows the formulation to continue to deliver the antimicrobial therapeutic agent and any secondary agents over a sustained time, eliminating the need for multiple applications. In another embodiment, the film forming agent is strong enough to protect against moisture and humidity incursion, yet flexible enough that the dried film is able to be peeled away by the patient before the next application of the therapeutic formulation, without any need to use a removing agent. In one embodiment, the film forming agent is present in an amount between about 10% and 85% by weight of the total formulation. In another embodiment, the amount of film forming agent in the formulations will be dependent upon the desired viscosity of the final formulation and the desired thickness of the film after application of the formulation. In one embodiment, the higher the concentration of the film forming agent, the higher the viscosity and the thickness of the film. In another embodiment, at a concentration of about 40% to about 80%, optionally 60% to about 70% of the film forming agent, the formulation after application results in a film upon drying that adheres to the site of infection, for example a nail, and acts as a non-occlusive membrane that allows the anti-microbial agent to be continuously delivered across the site of infection for a sustained-release effect, while protecting the infection from moisture and humidity.

In some embodiments, the therapeutic compounds described herein may be fabricated into the form of a sheet or a coating. For example, the therapeutic compounds described herein may be prepared to have antimicrobial properties and may be fashioned into a solid formulation for the treatment of burns or infected wounds as with a dressing, as inclusion into a coating for medical device or into a solid sheet protective component around a device where a breach in the skin may increase the likelihood of infection. As a component of a wound dressing, the therapeutic compounds may be associated with, but not limited to, natural biopolymers such collagen, gelatin, or biomedical materials that include polyurethanes, silicones, and hydrogels for example.

In other embodiments the presence of small amounts of sulfonated compounds as described herein in solid polymeric sheets can provide protection for protein therapeutic agents, such as insulin, from being degraded by enzymatic processes which can render these proteins ineffective and potentially pro-inflammatory to the site at which the drug is being delivered. In one example, a solid polymeric sheet containing polysulfonated compounds as described herein is fabricated from a biomedical material such as a silicone gel and the solid sheet is positioned to surround a transcutaneous access point through which a medical device such as an infusion set makes contact with a patient's tissue. The solid polymeric sheet, for example, can be formulated with an antibacterial, protease inhibiting, aqueous media soluble, polysulfonated material in salt form in order to protect the wound from invading microorganisms, thus preserving the viability of the subcutaneous tissue for the uptake of drug, and to preserve the drug by preventing degradation of the protein by proteases such a neutrophil elastase.

The compositions as described herein may additionally be provided in bottles or flasks from which they be dispensed by pouring, or by pumping such as via a manually pumpable trigger pump or manually operable trigger spray pump or using a suitable aerosolizing propellant. The therapeutic compositions can be provided and stored in a non-deformable bottle or in a squeezable container, such as a tube or deformable bottle which provides for easy dispensing of the composition by the consumer.

The therapeutic compounds described herein may additionally be part of a wound dressing device such as a biogel film, patch, dressing, bandage, wound covering, or other useful device for biomedical drug application, including, gauze, films, absorbtives, tape, wraps, bandages, hydrocolloids, hydrogels, alginates and collagen wound dressings, as well as materials and wound dressings described in U.S. patent application Ser. No. 13/797,864, U.S. patent application Ser. No. 13/483,643 (each incorporated herein by reference. The therapeutic compounds may be prepackaged with the wound dressings or applied to the wound dressings and then applied to the wound. The use of the therapeutic compounds as described herein applied to wounds for prolonged periods minimizes discoloration and long term cosmetic damage in comparison to other olidgodynamic invested wound dressings.

The sulfonated and thiolated therapeutic compounds described herein may additionally be associated and/or provided with any variety of pharmacologically active secondary cation agents including, antimicrobial, chemotherapeutic, antiseptic, antifungal, growth factors, antioxidant and/or analgesic agents.

Exemplary chemotherapeutic agents and other cancer fighting drugs include, but are not limited to, methotrexate, fluorouracil, adriamycin, ansamitocin, cytosine arabinoside, arabinosyl, adenine, mercaptopolylysine, PAM; L-PAM (phenylalanine mustard), mercaptopurine, mitotane, procarbazine dactinomycin (actinomycin D), mitomycin, plicamycin (mithramycin), aminoglutethimide, estramustine, flutamide, leuprolide, megestrol, tamoxifen, amsacrine (m-AMSA), asparaginase (L-asparaginase) Erwina asparaginase), etoposide (VP-16), interferon α-2a, interferon α-2b, teniposide (VM-26), adriamycin, arabinosyl, procarbazine, dacarbazine, Chlorambucil, Chlormethine, Cyclophosphamide, ifosfamide, Melphalan, Carmustine, Fotemustine, Lomustine, Streptozocin, Carboplatin, Oxaliplatin, BBR3464), Busulfan, Dacarbazine, Mechlorethamine, Procarbazine, Temozolomide, ThioTEPA, Uramustine, pemetrexed, raltitrexed, cladribine, clofarabine, fludarabine, mercaptopurine, thioguanine, capecitabine, cytarabine, gemcitabine, vinblastine, vincristine, vindesine, vinorelbine, daunorubicin, doxorubicin, epirubicin, idarubicin, mitoxantrone, valrubicin, bleomycin, mitomycin, topotecan, irinotecan, alemtuzumab, bevacizumab, cetuximab, gemtuzumab, panitumumab, rituximab, tositumomab, trastuzumab, etanercept, aminolevulinic acid, methyl aminolevulinate, porfimer sodium, verteporfin, dasatinib, erlotinib, gefitinib, imatinib, lapatinib, nilotinib, sorafenib, sunitinib, vandetanib (ZD6474), altretamine, anagrelide, bortezomib, denileukin diftitox, estramustine, pentostatin, pegaspargase, alagebrium (3-phenacyl-4,5-dimethylthiazolium; anti-helmintics; antitoxins; antivenins; aminoglycosides.

Exemplary antibiotics, include, but are not limited to aminoglycosides, amikacin, gentamicin, kanamycin, neomycin, netilmicin, tobramycin, paromomycin, spectinomycin, ansamycins, geldanamycin, herbimycin, rifaximin, streptomycin, carbacephem, loracarbef, carbapenems, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, clindamycin, lincomycin, lipopeptide, daptomycin, macrolides, azithromycin, clarithromycin, dirithromycin, erythromycin, roxithromycin, troleandomycin, telithromycin, spiramycin, monobactams, aztreonam, nitrofurans, furazolidone, nitrofurantoin, oxazolidonones, linezolid, posizolid, radezolid, torezolid, penicillins, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin/clavulanate, ampicillin/sulbactam, piperacillin/tazobactam, ticarcillin/clavulanate, bacitracin, colistin, polymyxin quinolones/fluoroquinolone, ciprofloxacin, enoxacin, gatifloxacin, gemifloxacin, levofloxacin, lomefloxacin, moxifloxacin, nalidixic acid, norfloxacin, ofloxacin, trovafioxacin, grepafloxacin, sparfloxacin, temafloxacin, sulfonamides, mafenide, sulfacetamide, sulfadiazine, sulfadimethoxine, sulfamethizole, sulfamethoxazole, sulfanilimide, sulfasalazine, sulfisoxazole, trimethoprim-sulfamethoxazole(co-trimoxazole) (tmp-smx), sulfonamidochrysoidine, tetracyclines, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

Exemplary local anesthetics, include, but are not limited to, mylocaine, articaine, benzocaine, benzonatate, bupivacaine, butacaine, butanilicaine, chloroprocaine, cinchocaine (dibucaine), dimethocaine (larocaine), etidocaine, eucaine, hexylcaine, levobupivacaine, lidocaine (lignocaine), mepivacaine, meprylcaine, metabutoxycaine, orthocaine, oxetacaine (oxethazaine), oxybuprocaine (benoxinate), phenacaine, piperocaine (metycaine), pramocaine (pramoxine), prilocaine, procaine, proparacaine (proxymetacaine), propoxycaine, quinisocaine (dimethisoquin), ropivacaine, tetracaine (amethocaine), and trimecaine.

Exemplary anti-fungals, include, but are not limited to, amphotericin b, candicidin, filipin, hamycin, natamycin, nystatin, rimocidin, clotrimazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox olamine, 5-fluorocytosine, griseofulvin, haloprogin, polygodial, tolnaftate, undecylenic acid, crystal violet, oregano, allicin, citronella oil, coconut oil, iodine, lemon myrtle, neem seed oil, olive leaf, orange oil, palmarosa oil, patchouli, selenium, tea tree oil, zinc, horopito, turnip, chives, radish, and garlic. Growth factors including, but not limited to, EGF (epidermal growth factor), PDGF (platelet-derived growth factor), TGF-α (transforming growth factor alpha), TGF-β1 (transforming growth factor beta), KGF or FGF-7 (keratinocyte growth factor), aFGF or FGF-1 (fibroblast growth factor), bFGF or FGF-2 (fibroblast growth factor) VEGF/VEP (vascular endothelial growth), CTGF (connective tissue growth factor), GM-CSF or CSF α, TNFα (tumor necrosis factor alpha), IL-1β (interleukin 1 β), IL-8 (interleukin 8).

Exemplary analgesics include, but are not limited to, benzocaine, camphor, capsaicin, diphenhydramine, hydrocortisone, lidocaine, menthol, methyl salicylate, pramoxine, nsaids, cox-2 inhibitors, and opioids.

Exemplary antiseptics, include, but are not limited to acetic acid, cadexomer idine, cetrimide, chlorhexidine glutonate, hexachlorophene, iodine compounds, providine compounds, sodium chlorite, hydrogen peroxide, silver nitrate and mebromin.

Exemplary anti-inflammatory agents include, but are not limited to, hydrocortisone, hydroxyltriamcinolone, alphamethyldexamethasone, dexamethasone-sodium phosphate, dexamethasone; beclomethasone dipropionate, clobetasolvalerate, desonide, desoxymethasone, desoxycorticosterone acetate, dexamethasone, dichlorisone, diflorasonediacetate, diflucortolonevalerate, fluadrenolone, fluclaroloneacetonide, fludrocortisone, flu methasonepivalate, fluosinoloneacetonide, fluocinonide, flucortinebutylester, fluocortolone, fluprednidene (fluprednylidene)acetate, flurandrenolone, halcinonide, hydrocortisone acetate, hydrocortisone butyrate, methylprednisolone, triamcinoloneacetonide, cortisone, cortodoxone, flucetonide, fludrocortisone, difluorosonediacetate, fluradrenaloneacetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasonedipropionate, betamethasonedipropionate, triamcinolone prostaglandin H synthetase inhibitors (Cox I or Cox II), flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam, carprofen, cyclooxygenase type II selective inhibitors, vioxx, celecoxib, P54, etodolac, L-804600, S-33516; PAF antagonists, A-137491, ABT-299, apafant, bepafant, minopafant, E-6123, BN-50727, nupafant, modipafant, PDF IV inhibitors, ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and/or roflumilast.

Exemplary anti-proteolytic agents include but are not limited to, amprenavir (Agenerase), fosamprenavir (Lexiva), indinavir (Crixivan), lopinavir/ritonavir (Kaletra), ritonavir (Norvir), saquinavir (Fortovase), and nelfinavir (Viracept), salts of ethylene diamine tetracetic acid, salts of polystyrene sulfonate, and sulfated oligo & polysaccharides.

In addition, other antimicrobial agents may be used with or combined with the therapeutic compounds described herein. Such antimicrobial agents include, but are not limited to, chloroxylenol (parachlorometaxylenol), acedapsone; acetosulfone sodium; alamecin; alexidine; amdinocillin; amdinocillin; pivoxil; amicycline; amifloxacin; amifloxacinmesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin, methylenedisalicylate; bacitracin zinc; bambermycins; benzoylpas calcium; berythromycin; betamicin sulfate; biapenem; biniramycin; biphenamine hydrochloride; bispyrithionemagsulfex; butikacin; butirosin sulfate; capreomycin sulfate; carbadox; carbenicillin disodium; carbenicillin, indanyl sodium; carbenicillin phenyl sodium; carbenicillin potassium; carumonam sodium; cefaclor; cefadroxil; cefamandole; cefarnandolenafate; cefamandole sodium; cefaparole; cefatrizine; cefazaflur sodium: cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; ccfpodoxime; proxetil; cefprozil; cefroxadine; cefsulodin sodium; ceftazidime; ceftibuten; ceftizoxime sodium; ceftriaxone sodium; cefuroxime; cefuroximeaxetil; cefuroximepivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicolpalmitate; chloramphenicolpantothenate complex; chloramphenicol sodium succinate; chlorhexidinephosphanilate; chlorhexidinediacetate, chlorhexidinedihydrochloride, chlorhexidinedigluconate, chlortetracycline bisulfate; chlortetracycline hydrochloride; cinoxacin; ciprofloxacin; ciprofloxacin hydrochloride; cirolernycin; clarithromycin; clinafloxacin hydrochloride; clindamycin; clindamycin hydrochloride; clindamycinpalmitate hydrochloride; clindamycin phosphate; clofazimine; cloxacillinbenzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycyclinefosfatex; doxycyclinehyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; fludalanine; flumequine; fosfomycin; fosfomycintromethamine; fumoxicillin; furazolium chloride; furazoliumtartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocyclinesulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensinsodiumr; monovalent silver salts, nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; octenidinedihydrochloride, octenidinediacetate, octenidinedigluconate, ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacinmesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillinpamoate; pivampicillinprobenate; polyhexamethylenebiguanide (polyhexanide hydrochloride, PHMB); polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicinstearate; rosoxacin; roxarsone; roxithromycin; sancycline; sanfetrinem sodium; sarmoxicillin; sarpicillin; scopafungin; silver acetate; silver nitrate, nanocrystalline silver, silver polystyrene sulfonate (“cross-linked” and non-cross-linked); silver carboxymethyl cellulose, silver polysaccharides (such as silver chondroitin sulfate and the like), silver carbene compounds, sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadiazine silver; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazolediolamine; sulfomyxin; sulopenem; sultarnricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillincresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and/or zorbamycin.

In some embodiments, the therapeutic compounds described herein may be part of a multi-step wound treatment process directed to assisting the various stages of the wound healing process. For example, in cases of severe trauma such as burns, there is a hyper-metabolic and hyper-inflammatory response that can damage healthy tissue as well as delay the healing process. The therapeutic compounds as described herein may be assembled as part of a multistage treatment proves to address the hyper-metabolic and hyper-inflammatory response of severe trauma. In a first step, the wound may be treated with sulfonated therapeutic compounds as described herein with antimicrobial and anti-protease activity. Such compounds may additionally be growth factor protective. The sulfonated therapeutic compounds may be applied alone or in combination with secondary therapeutic agents including antimicrobial, antifungal, antibiotic, antiseptic, anti-inflammatory, analgesic, an/or antioxidant agents. Protease levels may be monitored by any means generally known by those of skill in the art including point of care tests or optical sensors. The sulfonated therapeutic compounds may be applied one or more times as needed until the protease levels in the wound reach normal levels for wound healing. Once the wound reaches normal levels, a second treatment step may apply antimicrobial therapeutic compounds as described herein which do not contain anti-protease activity. For example, a thiolated oligodynamic metal composition such as silver GSH may be applied. In other embodiments, the second treatment step may comprise one or more secondary therapeutic agents including antimicrobial, antifungal, antibiotic, antiseptic, anti-inflammatory, analgesic, an/or antioxidant agents.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list.

The following examples are provided to illustrate the invention, but are not intended to limit in any way the scope of the invention.

### Example I

### Preparation of PSS-Ag

Sodium Polystyrene Sulfonate (SPSS, 70,000 mw) acquired from Sigma-Aldrich, St. Louis, Mo. and purified by precipitation from a 20-25% solution, was dissolved into deionized water to yield a 10-20% solids solution. Separately, silver acetate (Fluka) was suspended into deionized water, stirred, and the mixture heated to 60° C. while maintaining stirring. To the stirring mixture, 100 g of Dowex® Marathon strong cation exchange resin (Dow Chemical) was added to the mixture and dissolution/disappearance of the silver acetate immediately followed. The Ag+ modified Dowex® Marathon strong cation exchange resin, which is in fact a water-insoluble salt, can be isolated, washed with deionized water and dried. The Ag+ modified cation exchange resin is photo sensitive and should be stored away from room light. The dried, Ag+ modified Dowex® Marathon strong cation exchange resin is added to the 10-20% solution of sodium polystyrene sulfonate (SPSS) in a jar with a PTFE lined cap; the jar is scaled and placed onto a roller mill for about one hour. Subsequently, the Dowex® resin is filtered from the solution, washed with deionized water and the resulting solution is placed into a lyophilizer vessel, the solution is frozen, and the lyophilizer vessel containing the frozen material is connected to the lyophilizer until only a powder remains. The percentage of silver incorporation onto the PSS backbone is dependent upon the relative excess of silver-modified Dowex® resin utilized. Adjustment (lowering) of the ratio of silver to sodium in the silver-modified PSS can be accomplished by blending solutions of sodium polystyrene sulfonate (SPSS) and PSS-Ag (with Ag substitution up to 100%) and lyophilizing the equilibrated solution. Note: A 20% substituted PSS-Ag as determined by atomic absorption (20% Ag, 80% Na) can be combined with an equimolar amount of SPSS in solution and the solution lyophilized to yield a 10% Ag substituted PSS.

### Example III

### Synthesis of Silver Polystyrene Sulfonate (PSS-Ag, Quantitative Exchange)

10 g of dried PSS-H+ was added to a minimal volume of water (approximately 30 mL) and solubilized with a mechanical mixer. To the solution, 7.2 g of AgOAc (silver acetate) was added and mixed thoroughly. When the AgOAc is fully in solution, the reaction is completed. Then, 100% ethyl alcohol was added dropwise via an addition funnel and PSS-Ag⁺ precipitated out. The mixture was then allowed to settle and the solution was decanted off of the top of the precipitated and settled PSS-Ag⁺. The PSS-Ag+ was then allowed to air dry and was collected.

### Example III

### Preparation PSS-Arginine

Sodium Polystyrene Sulfonate (SPSS, 70,000 mw) acquired from Sigma-Aldrich, St. Louis, Mo. and purified by precipitation from a 20-25% solution in deionized water into isopropanol, was dissolved into deionized water to yield a 10-25% solids solution. Separately, arginine base in deionized water (10 mg/ml) was added to account for an equimolar quantity of sodium polystyrene sulfonate (SPSS), and the solution stirred for 1 hour at room temperature. The solution was transferred into a lyophilizer container and frozen. The frozen mass was placed under vacuum of the lyophilizer and the solid polystyrene arginine sulfonate was isolated as a flocculent off-white solid.

### Example IV

### Preparation of PSS-Mafenide

Sodium Polystyrene Sultanate (SPSS, 70,000 mw) acquired from Sigma-Aldrich, St. Louis, Mo. and purified by precipitation from a 20-25% solution in deionized water into isopropanol, was dissolved into deionized water to yield a 10-25% solids solution. Separately, 4-aminomethylbenzenesulfonamide acetate was dissolved into deionized water (10 mg/ml) and was added to account for an equimolar quantity of sodium polystyrene sultanate (SPSS), and the solution stirred for 1 hour at room temperature. The solution was transferred into a lyophilizer container and frozen. The frozen mass was placed under vacuum of the lyophilizer and the solid polystyrene mafenide sulfonate was isolated as a flocculent off-white solid.

### Example V

### Formation of Silver poly(4-styrenesulfonate)

Poly(4-styrenesulfonic acid) solution (MW ˜75,000, 18 wt. % in H2O, Sigma-Aldrich #561223 St. Louis, Mo.) (PSS-H) was concentrated via lyophilization from 18 wt. % to 36 wt. %. 88.8365 g of AgOAc (anhydrous, 99% pure, Alfa Aesar, #11660) was added to a vessel containing 100 g of PSS-H (277.7778 g of 36 wt. % PSS-H aqueous solution). The mixture was stirred with a stir-plate or overhead mixer for 30 minutes at room temperature (˜20° C.) and a strong acetic acid smell was evident. The resulting solution was then added drop wise to 2 L of 100% alcohol (reagent grade 90% ethanol, 5% methanol, 5% isopropanol) under high shear and stirred for 5 minutes until a polymer precipitated. The precipitated polymer was then filtered under vacuum and the solid washed 4× with 100% alcohol until no odor was detectable. The precipitate was then collected and dried in a vacuum oven for 4-18 hours at 60° C. The PSS-Ag was stored in a dry amber or opaque bottle at or below 20° C.

### Example VI

### Formation of Zinc (II) poly(4-styrenesulfonate)

Poly(4-styrenesulfonic acid) solution (MW ˜75,000, 18 wt. % in H2O, Sigma-Aldrich #561223 St. Louis, Mo.) (PSS-H) was concentrated via lyophilization from 18 wt. % to 36 wt. %. 88.8752 g of Zn(OAc)₂.2H₂O (dihydrate, ACS granular, Macron, #874004) was then added. The mixture was stirred with a stir-plate or overhead mixer for 30 minutes at room temperature (˜20° C.) and a strong acetic acid smell was evident. The solution was then added drop wise to 2 L of 100% alcohol (reagent grade 90% ethanol, 5% methanol, 5% isopropanol) under high shear and stirred for 5 minutes until a polymer precipitated. The precipitated polymer was filtered under vacuum and the solid washed 4× with 100% alcohol until no odor was detectable. The precipitated product was then dried in a vacuum oven for 4-18 hours at 60° C. and the PSS-Zn was stored in a dry amber or opaque bottle at or below 20° C.

### Example VII

### Formation of Copper (II) poly(4-styrenesulfonate)

Poly(4-styrenesulfonic acid) solution (MW 75,000, 18 wt. % in H2O, Sigma-Aldrich #561223 St. Louis, Mo.) (PSS-H) was concentrated via lyophilization from 18 wt. % to 36 wt. %. 73.5384 g Copper acetate (Cu(OAc)₂.H₂O) (monohydrate, laboratory grade, Ward's Science, #9410806) was then added to vessel containing 100 g of PSS-H. The mixture was stirred with a stir-plate or overhead mixer for 30 minutes at room temperature (˜20° C.) and a strong acetic acid smell was evident. The solution was then added drop wise to 2 L of 100% alcohol (reagent grade 90% ethanol, 5% methanol, 5% isopropanol) under high shear and stirred for 5 minutes until a polymer precipitated. The polymer was then filtered under vacuum and the solid washed 4× with 100% alcohol until no odor was detectable. The precipitated product was then dried in a vacuum oven for 4-18 hours at 60° C. The resulting PSS-Cu was stored in a dry amber or opaque bottle at or below 20° C.

### Example VIII

### Formation of Mafenide poly(4-styrenesulfonic acid)

Poly(4-styrenesulfonic acid) solution (MW 75,000, 18 wt. % in 1120, Sigma-Aldrich #561223) (PSS-H) was concentrated via lyophilization from 18 wt. % to 36 wt. %. 131.0884 g of mafenide acetate (MafOAc) (anhydrous, USP, Changzhou, #13009-99-9) was added to a vessel containing 100 g of PSS-H (277.7778 g of 36 wt. % PSS-H aqueous solution). The solution was stirred with a stir-plate or overhead mixer for 30 minutes at room temperature (˜20° C.) until a strong acetic acid smell was evident. The resulting solution was then added drop wise to 2 L of 100% alcohol (reagent grade 90% ethanol, 5% methanol, 5% isopropanol) under high shear and stirred for 5 minutes until the polymer precipitated. The precipitated polymer was filtered under vacuum and the solid washed 4× with 100% alcohol until no odor was detectable. The precipitated product was then collected and dried in a vacuum oven for 4-18 hours at 60° C. The PSS-Maf was stored in a dry amber or opaque bottle at or below 20° C.

### Example IX

### Formation of chlorhexidine poly(4-styrenesulfonate)

Poly(4-styrenesulfonic acid) solution (MW 75,000, 18 wt. % in H2O, Sigma-Aldrich #561223) (PSS-H) was concentrated via lyophilization from 18 wt. ° A to 36 wt. %. 166.4819 g of chlorhexidine diacetate (Chx(HOAc)₂) (Changzhou, #C6143) was then added to a vessel containing 100 g of PSS-H (277.7778 g of 36 wt. % PSS-H aqueous solution) and the mixture was stirred with a stir-plate or overhead mixer for 30 minutes at room temperature (˜20° C.) and a strong acetic acid smell was evident. The solution was then added drop wise to 2 L of 100% alcohol (reagent grade 90% ethanol, 5% methanol, 5% isopropanol) under high shear and stirred for 5 minutes until a polymer precipitated. The precipitated polymer was filtered under vacuum and the solid washed 4× with 100% alcohol until no odor was detectable. The precipitated product was then dried in a vacuum oven for 4-18 hours at 60° C. The PSS-Chx was stored in a dry amber or opaque bottle at or below 20° C.

### Example X

### Formation of Octenidine poly(4-styrenesulfonate)

Poly(4-styrenesulfonic acid) solution (MW ˜75,000, 18 wt. % in H2O, Sigma-Aldrich #561223) (PSS-H) was concentrated via lyophilization from 18 wt. % to 36 wt. %. 166.0162 g octenidine dihydrochloride (Oct(HCl)₂) (Dishman Pharmaceuticals & chemical LTD., #70775-75-6) was added to a vessel containing 100 g of PSS-H (277.7778 g of 36 wt. % PSS-H aqueous solution). The mixture was then stirred with a stir-plate or overhead mixer for 30 minutes at room temperature (˜20° C.) and a strong acetic acid smell was evident. The solution was then added drop wise to 2 L of 100% alcohol (reagent grade 90% ethanol, 5% methanol, 5% isopropanol) under high shear and stirred for 5 minutes and a polymer precipitated. The precipitated polymer was filtered under vacuum and the solid washed 4× with 100% alcohol until no odor was detectable. The precipitated product was dried in a vacuum oven for 4-18 hours at 60° C. The PSS-Oct was stored in a dry amber or opaque bottle at or below 20° C.

### Example XI

### Silver Polystyrene Sulfonate Ointment/Cream Formulation with Reduced Glutathione (GSH)

Silver polystyrene sulfonate (22.6 wt % silver, 49.82% substituted) was formulated into an oil-in-water emulsion/cream (Per 100 g: 1 gram of Allantoin, 25 grams of white petrolatum, 10 grams of Stearyl alcohol, 2 grams of Steareth 21 (Brij 721), 3 grams of Steareth 2 (Brij 72), 10 grams of propylene glycol, 0.02 grams of propylparaben, 0.17 grams of methylparaben, 8 grams of isopropyl myristate, 7 grams of polyoxyl 40 stearate, 33.8 grams of purified water) at 6.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration was shown to be effective at 1.5-3.0 wt % GSH (a ratio of approximately 2:1 silver to sulfhydryl) as evidenced by zones of inhibition, cytotoxicity, and photo stability.

### Example XII

### Silver Chondroitin Sulfate Ointment/Cream Formulation

Silver chondroitin sulfonate (14 wt % silver, 75% substituted) was formulated into an oil-in-water emulsion/cream (Per 100 g: 1 gram of Allantoin, 25 grams of white petrolatum, 10 grams of Stearyl alcohol, 2 grams of Steareth 21 (Brij 721), 3 grams of Steareth 2 (Brij 72), 10 grams of propylene glycol, 0.02 grams of propylparaben, 0.17 grams of methylparaben, 8 grams of isopropyl myristate, 7 grams of polyoxyl 40 stearate, 33.8 grams of purified water) at 6.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration was shown to be effective at 1.5-3.0 wt % GSH (a ratio of approximately 2:1 silver to sulfhydryl) as evidenced by zones of inhibition, cytotoxicity, and photostability.

### Example XIII

### Silver Polystyrene Sulfonate Ointment/Cream Formulation with L-Cysteine

Silver polystyrene sulfonate (22.6 wt % silver, 49.82% substituted) was formulated into an oil-in-water emulsion/cream (Per 100 g: 1 gram of Allantoin, 25 grams of white petrolatum, 10 grams of Stearyl alcohol, 2 grams of Steareth 21 (Brij 721), 3 grams of Steareth 2 (Brij 72), 10 grams of propylene glycol, 0.02 grams of propylparaben, 0.17 grams of methylparaben, 8 grams of isopropyl myristate, 7 grams of polyoxyl 40 stearate, 33.8 grams of purified water) at 6.0 wt % and L-cysteine at 0.25, 0.5, 1.0, and 3.0 wt % was added. The formulations were evaluated for color stability, antimicrobial effectiveness, and cytotoxicity and gauged against the control formulation without any cysteine. At least one effective concentration was shown to be 0.5-1 wt % L-cysteine (a ratio of approximately 1.5:1 silver to sulfhydryl) as evidenced by zones of inhibition, cytotoxicity, and photo stability.

### Example XIV

### Silver Polystyrene Sulfonate Gel Formulation with Reduced Glutathione (GSH)

Silver polystyrene sulfonate (22.6 wt % silver) was formulated into a water-miscible gel (Per 100 g of gel: 5 grams of Glycerine, 10 grams of Propylene Glycol, 1.5 grams of 2-hydroxyethyl cellulose (1,300,000 average MW) (Aldrich#434981), 4 grams of Hydroxypropylmethyl cellulose, 2910 (Methocel E4M Premium) 79.5 g of deionized deoxygenated sterile water) at 6.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration was shown to be 1.5 to 3.0 wt % GSH (a ratio of approximately 2:1 silver to sulfhydryl) as evidenced by zones of inhibition, cytotoxicity, and photostability.

### Example XV

### Silver Polystyrene Sulfonate Gel Formulation with L-Cysteine

Silver polystyrene sulfonate (22 wt % silver) was formulated into a water-miscible gel (Per 100 g of gel: 5 grams of Glycerine, 10 grams of Propylene Glycol, 1.5 grams of 2-hydroxyethyl cellulose (1,300,000 average MW) (Aldrich#434981), 4 grams of Hydroxypropylmethyl cellulose, 2910 (Methocel E4M Premium) 79.5 g of deionized deoxygenated sterile water) at 6.0 wt % and L-cysteine at 0.5, 1.0, and 3.0 wt % was added. The formulations were evaluated for color stability, antimicrobial effectiveness, and cytotoxicity and gauged against the control formulation without any cysteine. At least one concentration was shown to be 0.5-1 wt % L-cysteine (a ratio of approximately 2:1 silver to sulfhydryl) as evidenced by zones of inhibition, cytotoxicity, and photostability.

### Example XVI

### Silver Carbene Compounds in Ointment/Cream Formulation

Silver carbene compound was formulated into an oil-in-water emulsion/cream (Per 100 g: 1 gram of Allantoin, 25 grams of white petrolatum, 10 grams of Stearyl alcohol, 2 grams of Steareth 21 (Brij 721), 3 grams of Steareth 2 (Brij 72), 10 grams of propylene glycol, 0.02 grams of propylparaben, 0.17 grams of methylparaben, 8 grams of isopropyl myristate, 7 grams of polyoxyl 40 stearate, 33.8 grams of purified water) at 5.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration range was shown to be 1.5-3.0 wt % GSH (a ratio of approximately 2:1 silver to sulfhydryl) as evidenced by zones of inhibition, cytotoxicity, and photo stability.

### Example XVII

### Silver Carbene Compound in a Gel Formulation

Silver carbene was formulated into a water-miscible (Per 100 g of gel: 5 grams of Glycerine, 10 grams of Propylene Glycol, 1.5 grams of 2-hydroxyethyl cellulose (1,300,000 average MW) (Aldrich#434981), 4 grams of Hydroxypropylmethyl cellulose, 2910 (Methocel E4M Premium) gel at 5.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration was shown to be 1.5 to 3.0 wt % GSH (a ratio of approximately 2:1 silver to sulfhydryl) as evidenced by zones of inhibition, cytotoxicity, and photo-stability.

### Example XVIII

### Silver Sulfadiazine in a Cream Formulation

Silver sulfadiazine was formulated into an oil-in-water emulsion/cream (Per 100 g: 1 gram of Allantoin, 25 grams of white petrolatum, 10 grams of Stearyl alcohol, 2 grams of Steareth 21 (Brij 721), 3 grams of Steareth 2 (Brij 72), 10 grams of propylene glycol, 0.02 grams of propylparaben, 0.17 grams of methylparaben, 8 grams of isopropyl myristate, 7 grams of polyoxyl 40 stearate, 33.8 grams of purified water) at 1.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration was shown to be 1.5-3.0 wt % GSH (a ratio of approximately 2:1 silver to sulfhydryl) as evidenced by zones of inhibition, cytotoxicity, and photostability.

### Example XIX

### Silver Sulfadiazine in a Gel Formulation

Silver sulfadiazine was formulated into a water-miscible gel (Per 100 g of gel: 5 grams of Glycerine, 10 grams of Propylene Glycol, 1.5 grams of 2-hydroxyethyl cellulose (1,300,000 average MW) (Aldrich#434981), 4 grams of Hydroxypropylmethyl cellulose, 2910 (Methocel 1E4M Premium) at 1.0 wt % and reduced glutathione (GSH) at 0.5, 1.0, 1.5, 2.0, 2.5, 3.0, and 6.0 wt % was added. The formulations were evaluated for color stability, antimicrobial effectiveness, and cytotoxicity and gauged against the control formulation without any glutathione. At least one concentration was shown to be effective at 1.5 to 3.0 wt % GSH (a ratio of approximately 2:1 silver to sulfhydryl) as evidenced by zones of inhibition, cytotoxicity, and photostability.

### Example XX

### GSH Treated co-(poly(divinyl benzene)-poly(styrene sulfonate)) Silver Salt

In a flask 10 grams of co-(poly(divinyl benzene)-poly(styrene sulfonate)) silver salt, 7.5% Ag by weight was suspended in 50 cc of deionized water and 0.2 grams of GSH was added. Following dissolution of the GSH, the resin was allowed to stir for an additional 24 hours at room temperature. The resin was filtered and washed with deionized water and dried. In a flask 10 grams of co-(poly(divinyl benzene)-poly(styrene sulfonate)) silver salt, 7.5% Ag by weight was suspended in 50 cc of deionized water and L-cysteine was added. The formulations were evaluated for color stability, antimicrobial effectiveness, and cytotoxicity and gauged against the control formulation without any cysteine.

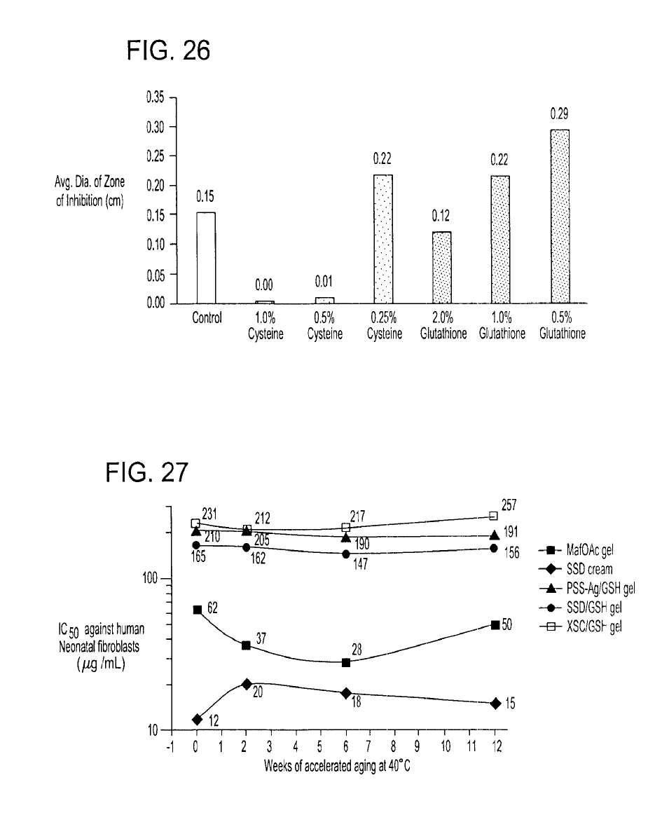

This procedure was repeated using several different glutathione or L-cysteine treatments at a variety of concentrations (0.25, 0.5, 1.0, 2.0 2.5, 3.0, 4.0, 5.0 wt %). The dried resins were evaluation using Kirby-Bauer zone of inhibition assays. As can be seen in FIG. 26, 0.5% glutathione was more effective than 0.5% cysteine in an average zone of inhibition taken from Pseudomonas aeruginosa, Acinetobacter baumannii, and Klebsiella pneumonia. Against methicillin-resistant Staphylococcus aureus, the largest zones were observed for resin formulations that were treated with GSH at about 2 wt %. This trend held for all of the formulations evaluated.

### Example XXI

### Effect of Silver Compounds on Bacterial Growth

Comparisons were made between varying concentrations of polystyrene sulfonate silver, polystyrene sulfonate silver with GSH, caffeine carbene silver, caffeine carbene silver with GSH, pyrimidine silver carbene and pyrimidine silver carbene with GSH and their effect on Pseudomonas aeruginosa, Acinetobacter baumannii, and Klebsiella pneumonia.

Silver formulations containing 5.0 wt % of the active compound (polystyrene sulfonate silver, caffeine carbene silver, and pyrimidine silver carbene) with 2.0% GSH where indicated were added to 100 μL of tryptic soy broth supplemented with 10% fetal bovine serum in wells of a sterile 96 well plate, one plate for each type of bacteria to avoid cross contamination. (Incubator set at 37° C. and shaking at 220 rpm) 10⁴ of each bacteria (determined by standard curve of OD600 nm vs. CFU) diluted in 100 μL of tryptic soy broth supplemented with 10% fetal bovine serum was added to the wells. The OD600 nm levels were monitored until OD600 nm of untreated control reached an OD600 nm of approximately 1.0.

The OD600 nm for all wells was collected and the MIC₉₀ was calculated by interpolating a linear fit of the linear portion of the curve of the plot of compound concentration versus OD600 nm to find the concentration at which the OD600 nm is 10% of its maximum value.

As shown in FIGS. 1, 2, and 3, silver compounds with GSH were better at inhibiting the growth of Pseudomonas aeruginosa, Acinetobacter baumannii, and Klebsiella pneumonia than the silver compounds alone. For example, as shown in FIG. 1, polystyrene sulfonate silver with GSH and caffeine carbene silver with GSH were more effective at lower concentrations at inhibiting Pseudomonas aeruginosa growth than polystyrene sulfonate silver and caffeine carbene silver alone. As shown in FIG. 2, silver sulfadiazine with GSH was significantly more effective in inhibiting the growth of Acinetobacter baumannii than silver sulfadiazine alone. As shown in FIG. 3, silver sulfadiazine with GSH was significantly more effective in inhibiting the growth of Klebsiella pneumonia growth than silver sulfadiazine alone.

### Example XXII

### Cytotoxicity of Formulations Using Human Neonatal Fibroblasts

Growth and Viability assays (5,000 and 10,000 cells/well respectively) were set up with human neonatal fibroblasts in sterile tissue culture treated 96 well plates in DMEM (Invitrogen #11995) supplemented with 10% fetal calf serum (Invitrogen #10437-028) 1% Antibiotic-Antimycotic (Invitrogen #15240-062). (Incubator set at 37 C and 5% CO2). Cells were allowed to grow overnight, media was removed and replaced with media doped with desired concentration of polystyrene sulfonate silver, silver sulfadiazine, caffeine silver carbene, theobromine silver carbene and pyrimidine silver carbene (PSC) with and without GSH and cells were allowed to incubate with the sample overnight.

10 μL of prepared MTS reagent (Promega # G5430) was added to the wells and absorbance at 490 nm was read once color has stabilized (1-4 hours). IC₅₀ levels were determined by interpolating a linear fit of the linear portion of the curve of the plot of compound concentration versus OD490 nm to find the concentration at which the OD490 nm was 50% of its maximum value.

As shown in FIGS. 4 and 5, once the concentration of the silver compounds reached 10 μg/mL, the cytotoxicity of the compounds with GSH was less than the compositions without.

### Example XIII

### Therapeutic Index of Silver Compounds

Comparisons were made between xanthine based silver carbene (XSC), xanthine based silver carbene and reduced glutathione (G), polystyrene sulfonate silver, polystyrene sulfonate silver with reduced glutathione (G), silver sulfadiazine and glutathione, silver sulfadiazine, silver glutathione, and Ascend® Laboratories 1% SSD cream and their effect on Pseudomonas aeruginosa, Acinetobacter baumannii, and Klebsiella pneumonia.

Silver formulations containing 5.0 wt % of the active compound (between xanthine based silver carbene (XSC), polystyrene sulfonate silver, silver sulfadiazine, silver, and Ascend® Laboratories 1% SSD cream with and without GSH or reduced glutathione where indicated were added to 100 μL of tryptic soy broth supplemented with 10% fetal bovine serum in wells of a sterile 96 well plate, one plate for each type of bacteria to avoid cross contamination. (Incubator set at 37° C. and shaking at 220 rpm) 10⁴ of each bacteria (determined by standard curve of OD600 nm vs. CFU) diluted in 100 μL of tryptic soy broth supplemented with 10% fetal bovine serum was added to the wells. The OD600 nm levels were monitored until OD600 nm of untreated control reached an OD600 nm of approximately 1.0.

The OD600 nm for all wells was collected and the MIC₉₀ was calculated by interpolating a linear fit of the linear portion of the curve of the plot of compound concentration versus OD600 nm to find the concentration at which the OD600 nm is 10% of its maximum value.

Growth and Viability assays (5,000 and 10,000 cells/well respectively) were set up with human neonatal fibroblasts in sterile tissue culture treated 96 well plates in DMEM (Invitrogen #11995) supplemented with 10% fetal calf serum (Invitrogen #10437-028) 1% Antibiotic-Antimycotic (Invitrogen #15240-062). (Incubator set at 37 C and 5% CO2). Cells were allowed to grow overnight, media was removed and replaced with media doped with xanthine based silver carbene (XSC), xanthine based silver carbene and reduced glutathione (G), polystyrene sulfonate silver, polystyrene sulfonate silver with reduced glutathione (G), silver sulfadiazine and glutathione, silver sulfadiazine, silver glutathione, and Ascend® Laboratories 1% SSD cream and cells were incubated with the sample overnight.

10 μl of prepared MTS reagent (Promega # G5430) was added to the wells and absorbance at 490 nm was read once color has stabilized (1-4 hours). IC₅₀ levels were determined by interpolating a linear fit of the linear portion of the curve of the plot of compound concentration versus OD490 nm to find the concentration at which the OD490 nm was 50% of its maximum value.

The therapeutic index was calculated as the IC₅₀/MIC₉₀. The therapeutic indices are shown in FIGS. 17-25 and summarized in Table 6 where the circles offer a comparison of therapeutic index values where greater shading indicates a greater TI value.

TABLE 6
Therapeutic Index (T.I.)

| | Pseudo | | MRSA | | AcBc | | Kleb | |
|---|---|---|---|---|---|---|---|---|
| | T.I. | | T.I. | | T.I. | | T.I. | |
| XSC/G | ◯ | 1.3 | ◯ | 0.4 | ◯ | 1.3 | ◯ | 0.7 |
| XSC | ◯ | 0.3 | ◯ | 0.2 | ◯ | 0.2 | ◯ | 0.2 |
| PSS-Ag/G | [symbol] | 4.8 | [symbol] | 1.9 | [symbol] | 4.0 | [symbol] | 3.8 |
| PSS-Ag | ◯ | 0.7 | ◯ | 0.3 | ◯ | 0.3 | ◯ | 0.4 |
| SSD/G | | 7.5 | [symbol] | 3.5 | | 6.4 | [symbol] | 5.9 |
| SSD | ◯ | 0.4 | ◯ | 0.4 | ◯ | 0.4 | ◯ | 0.2 |
| GSH-Ag | [symbol] | 2.9 | ◯ | 1.5 | [symbol] | 2.9 | ◯ | 1.4 |
| Ascend | ◯ | 1.0 | ◯ | 0.2 | ◯ | 0.3 | ◯ | 0.2 |

### Example XXIV

### Effectiveness of SSD:GSH Formulations in a Biofilm Model

Bacteria in infected wounds are likely to be predominately present as biofilms. Bacteria in biofilms are inherently more resistant to antimicrobial therapies than their planktonic counterparts. Biofilms of P. aeruginosa and K. pneumoniae are generated by seeding 24-well Transwell dishes with 10⁶ CFU in 500 μl of appropriate medium and incubating for 48 hours at 37° C. The medium is then removed by gentle aspiration and the biofilm washed with PBS to remove planktonic cells. Fresh medium containing half-log serial dilutions of polystyrene sulfonate silver, polystyrene sulfonate silver with GSH, caffeine carbene silver, caffeine carbene silver with GSH, pyrimidine silver carbene and pyrimidine silver carbene with GSH are then added to the insert plates in triplicate. The plates are then incubated for an additional 24 hours without movement. The inserts and bacterial medium are then removed by gentle aspiration and the biofilm again washed with PBS. Fresh broth is added and the biofilms disrupted by pipetting and agitation. Changes in the turbidity (absorbance at 600 nm) of the resuspended biofilm are monitored with time and compared to the same bacteria from biofilms treated with negative (no treatment or non-antimicrobial gel) and positive (e.g., Silvadene′) controls. At the conclusion of this aim, the optimal formulations as determined from BI measurements are chosen.

### Example XXV

### Inhibition of Elastase

About 15 g of Cutinova amorphous hydrogel (Beiersdorf A G, Unnastraβe 48, D-20245, Hamburg, Germany) was transferred to a vial and 1.67 g of PSS (mw=70,000) (Sigma-Aldrich, St. Louis, Mo.) was added and stirred into the gel using a glass rod to yield about a 10% (wt./wt.) solids composition. As shown in FIGS. 6 and 7, in the PSS-formulation 35 milliunits of elastase has been decreased to 6 milliunits by the Na-PSS (70K) formulation equating to about an 80% reduction of the elastase from the test sample (human wound fluid). This is significantly better than the Promogran gel (collagen and oxidized regenerated cellulose) and the gauze or polyester dressings alone.

### Example XXVI

### Dose Response Study of Sodium Polystyrene Sultanate (SPSS) Inhibition of Neutrophil Elastase

Different amounts of SPSS were incubated with 12.5 milliunits of neutrophil elastase in 1.0 ml of buffer for 2 hours at 25° C. Aliquots (160 microliters) were mixed with an elastase specific substrate and the liberation of anilide was monitored spectrophotometrically. Inhibition was rapid and irreversible with the full inhibitory effects observed as fast as the measurements could be made (−5 minute intervals). The rate of inhibition is a consequence of the inhibitor and the proteins being in the solution phase. Dilutions of elastase were used to generate a standard curve and concentrations down to 1 microgram/ml inhibited elastase efficiently (−80%) but not completely. As shown in FIG. 8, a comparison of SPSS against condroitin-6-sulfate (C6S) for the inhibition of elastase revealed that C6S was much less efficient an inhibitor than SPSS. C6S could not achieve the same level of inhibition (only 45%) and it required about 21 times the amount of C6S as SPSS to achieve an equivalent level of inhibition.

### Example XXVII

### Inhibition of MMP-9

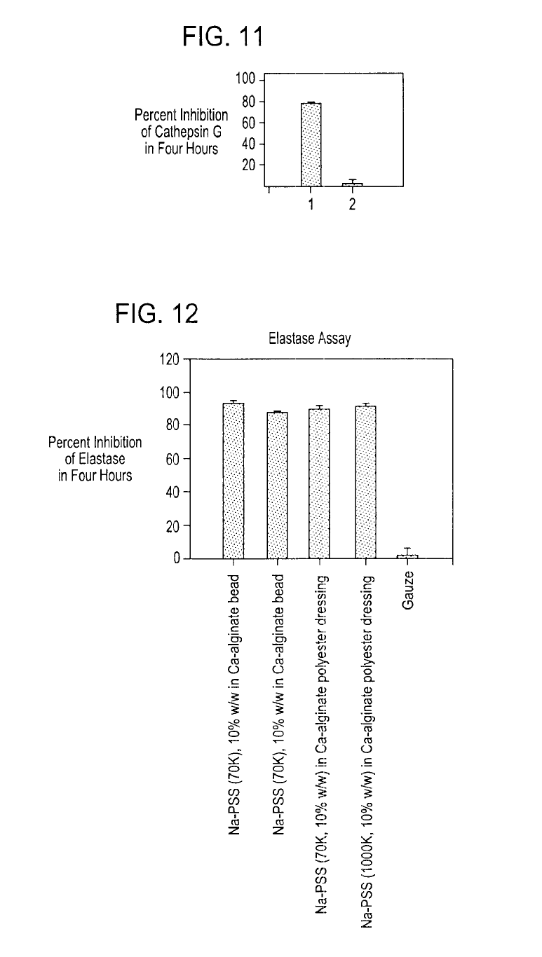

About 15 g of Cutinova amorphous hydrogel (Beiersdorf A G, Unnastraβe 48, D-20245, Hamburg, Germany) was transferred to a vial and 1.67 g of PSS (mw=70,000 and mw=1000K) (Sigma-Aldrich, St. Louis, Mo.) was added and stirred into the gel using a glass rod to yield about a 10% (wt./wt.) solids composition. As shown in FIGS. 9, 10, and 11, the gel formulation and the solid SPSS materials were effective inhibitors of the serine proteases cathepsin G, and the metalloproteases MMP-8 and MMP-9 in comparison to sulfonated SIBA, curafil wound gel (Covidien, Mansfield, Mass.), cutinova hydrogel (Smith and Nephew, St. Petersburg, Fla.) and gauze.

### Example XXVIII

### Formation and Use of Na-PSS Coated Calcium Alginate Beads

About 25 grams of sodium alginate (Sigma-Aldrich, PO Box 14508, St. Louis, Mo.) was combined 5 with 250 mL of sterile deionized water (as by autoclave sterilization) and the mixture was autoclaved in order to facilitate dissolution. Simultaneously 15 grams of PSS (1000K) were combined with 200 mL of sterile deionized water and autoclaved (to 105° C.) as the above described solution in order to facilitate dissolution. Following autoclaving the above solutions were combined and filtered through polyester fabric in order to remove non-dissolved matter. The combined solution was autoclaved one more time (105° C.) and capped in order to ensure shelf life. Separately, 1 liter of 0.5M CaCl₂ was prepared and 10 grams of PSS (1000K) were added in order to ensure that little SPSS was lost in the crosslinking step. The alginate solution was then added dropwise to the calcium chloride PSS solution in order to prepare beads. The beads were allowed to dwell in the calcium chloride solution for 5 minutes and subsequently filtered through polyester fabric. As shown in FIG. 12, the calcium PSS-containing beads were effective at inhibiting elastase.

### Example XXIX

### Formation of Na-PSS Coated Dressings

About 25 grams of sodium alginate (Sigma-Aldrich, St. Louis, Mo.) was combined 5 with 250 mL of sterile deionized water (as by autoclave sterilization) and the mixture was autoclaved in order to facilitate dissolution. Simultaneously 15 grams of PSS (1000K) were combined with 200 mL of sterile deionized water and autoclaved (to 105° C.) as the above described solution in order to facilitate dissolution. Following autoclaving the above solutions were combined and filtered through polyester fabric in order to remove non-dissolved matter. The combined solution was autoclaved one more time (105° C.) and capped in order to ensure shelf life. Separately, 1 liter of 0.5M CaCl₂ was prepared and 10 grams of PSS (1000K) were added in order to ensure that little SPSS was lost in the crosslinking step. A sheet of Evolon 130 (gr) soft (Freudenberg/Evolon NA) was then immersed in the alginate solution so as to become fully wetted and the fabric removed and excess alginate solution removed. The fabric was then placed into the CaCl₂-PSS solution and allowed to dwell until the alginate became firm. The fabric composite was cut to size and sterilized by electron beam irradiation (25 kG) prior to studies. As shown in FIG. 12, the Ca-alginate polyester dressing was effective in inhibiting elastase.

### Example XXX

### SPSS Microspheres for Controlled Release of SPSS Using a Water/Oil/Water Emulsion Technique

Sodium Polystyrene Sulfonate (SPSS, 70,000 mw Sigma-Aldrich, St. Louis, Mo.) was purified by precipitation from a 20-25% solution in deionized water into isopropanol was dissolved into deionized water to prepare about an 8% w/w solution. Separately, PLGA (50:50, Lactel Polymers, Cuppertino, Calif.) dissolved into dichloromethane to yield a solution of 5% solids. The two solutions were combined (with the total SPSS added representing 8% of the PLGA mass and shaken to form an emulsion. Separately, a 1% polyvinyl alcohol (PVA, 87-89% hydrolyzed) solution was prepared (roughly 30 times the total volume of the (PLGA) dichloromethane-aqueous (SPSS) emulsion. The emulsion was added to the rapidly stirring PVA solution and the mixture slowly heated to 45° C. The mixture was allowed to stir at 45° C. for 60 minutes and the mixture cooled and filtered. The tan colored material in the filter was washed with D-mannitol (2% w/w) and the spheres allowed to dry.

The microspheres were imaged on a hemocytometer grid and found to be very regular in shape and size considering that the experiment was carried out using standard laboratory equipment with few controls in place. An evaluation of 25 randomly chosen microspheres from the sample revealed that a mean spherical surface area of 0.0259 mm² (r=45 μm), a median of 0.0258 mm² (r=45 μm), and a std. dev. of 0.0123 mm². The minimum and maximum surface areas in the sample were found to be 0.00859 (r=26 urn) and 0.0492 mm² (r=63 μm) respectively. These surface areas translate to diameters of ca. 90 μm (mean), 52 μm (min) and 126 μm (max). Overall, these data detail a uniform process.

The release of SPSS from the microspheres was followed via UV spectroscopy and total release was shown to require about 5 days in PBS at 37° C.

### Example XXXI

### PSS Silver Ointment in the Treatment of Full Thickness Burns in the Rat (Comb Burn Model)

A three pronged brass template heated to 100° C. was used to generate three full thickness burns (1×2 cm) on each side of the spine of Wistar rats (n=G). Each of the burns are separated by two interspaces (0.5×2 cm). Escharotomies were created in the burn sites and the wounds were dressed with an ointment containing 14% Ag-PSS. Contralateral (control) wounds were dressed with vehicle ointment only. The Ag-PSS ointment preserved approximately twice the interspace tissue of the control ointment.

### Example XXXII

### Mafenide Acetate Treated Wounds in White Pig Model

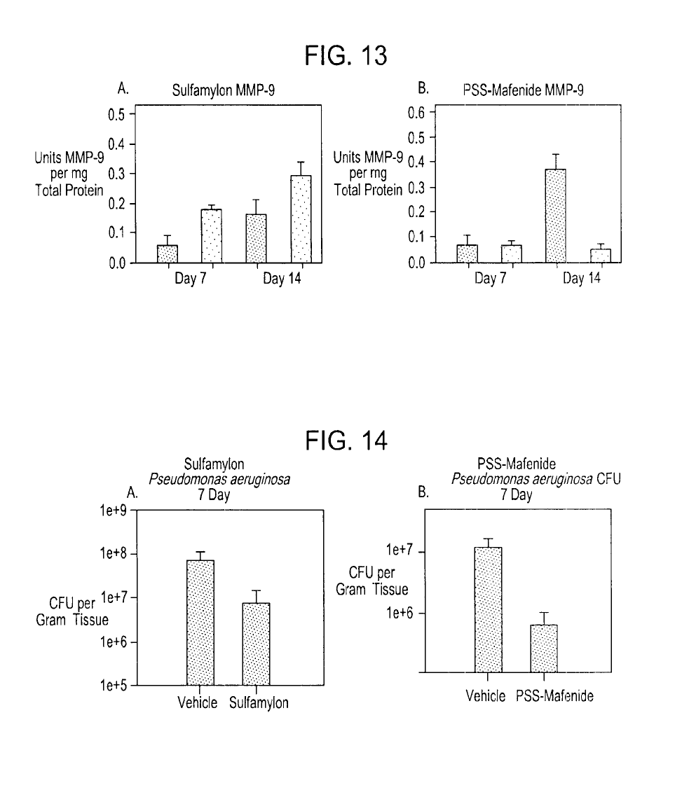

Observations were carried out on two white pigs, aged 15-16 weeks and weighing approximately 40 kg. Under general anesthesia, with ketamine hydrochloride and sodium thiopental-18 burn wounds were produced on the surface of the back—nine on each side. The surface of each wound was 15×30 mm. The total area of burns did not exceed 10% of total skin surface. On the first pig, 9 of the wounds were treated with 8.5% mafenide acetate cream (sulfamylon) or cream alone. On the second pig, wounds were treated with 8.5% w/w mafenide acetate plus Na-PSS or cream alone. Each wound was dressed with an aseptic dressing which was changed once a day and contained a comparable amount of therapeutic agent. As shown in FIG. 13, there was a higher level of MMP-9 expression on days 7 and 14 in wounds treated with mafenide acetate cream than cream alone. However, as shown in FIG. 14, in the wounds treated with mafenide acetate and Na-PSS, there was a significant decrease in MMP-9 expression compared to the results with cream alone.

### Example XXXIII

### Mafenide Acetate Treatment of Pig Burn Wounds Infected with Pseudomonas aeruginosa

Two young, female, specific pathogen-free pigs (SPF: Ken-O-Kaw Farms, Windsor. Ill.) weighing between 25 and 35 kg were used in this study. These animals were fed a non-antibiotic chow ad libitum before the study, fasted overnight before the procedures, and housed individually in animal facilities with controlled temperature (19-21° C.) and controlled light and dark cycles (12 hour light/12 hour dark). In order to minimize possible discomfort, analgesics (buprenorphine and fentanyl transdermal patches) were used during the entire experiment. Forty eight (48) partial thickness wounds were made on the paravertebral area of each animal. The wounds were then divided into 6 groups, each containing 8 wounds. 4 groups were inoculated with P. aeruginosa, and one group on each pig remained un-infected as a control. Two groups one on each animal were treated with cream vehicle. Two groups were treated with 8.5% mafenide acetate cream and two groups were treated with 8.5% mafenide acetate plus Na-PSS. As can be seen in FIG. 14, PS-Mafenide decreased the number of colony forming units over vehicle or 8.5% mafenide acetate cream alone.

### Example XXXIV

### Treatment of Wounds in Rats

Twenty four rats are divided into six groups for a total of 24 total animals. Under inhalant anesthesia, two pairs of paravertebral full thickness wounds (1.0×1.0 cm) are generated in Wistar rats (n=4 wounds/animal). The periphery of each wound is tattooed and in each animal, one wound will be treated with a candidate SSD:GSH, a second wound will covered with a dressing (e.g., Tegaderm®, 3M) only, a third wound will be treated with vehicle sans SSD:GSH, and the fourth wound with a commercial form of w/w SSD (e.g. Silvadene®). Dressing changes are performed every day. At seven days post wounding, animals are sacrificed, wounds photographed, biopsied, and specimens fixed for histology. The absence of adverse effects (e.g., erythema, macular/papular eruption, ulcerating dermatitis, symptoms of general illness, lack of healing) is used as an indication of low toxicity. Healing parameters include quantifying re-epithelization, contraction (via histopathology), and determining the amount and state of granulation tissue with the aid of staining for Von Willebrand factor (stored in the in the Weibel-Palade bodies of the endothelium) to identify blood vessels.

The bacterial load is quantified by taking biopsy specimens (6 mm trephine punch) weighing them, and homogenizing them in PBS using a Brinkman Polytron. The homogenate is serially diluted and plated onto Nutrient agar plates. After incubating at 37° C. for 24 hr, the numbers of total CFU/gram tissue is then calculated. Replicative plating to differential/selective media (MacConkey) is used to identify P. aeruginosa colonies and K. pneumoniae colonies. Because of the size of the initial inoculum, interference from endogenous organisms is not expected to be significant if even detectable through day 7. If present, endogenous organisms will be included in total.

Rates of healing are determined by rates of re-epithelialization, granulation tissue formation, and contraction. Planimetry is used to determine the percent change in wound areas. Re-epithelization is calculated by measuring epithelial tongues in computer images of H&E stained sections. Re-epithelialization (%) is determined by summing the length of the two tongues, dividing by the initial wound size rather than dividing by the distance between the tattoo marks (location of the initial wound edge), and multiplying by 100. This minimizes error introduced by contraction that may have occurred during healing.

The extent of inflammation is assessed measuring myeloperoxidase (MPO) activity. MPO is a standard marker for neutrophils (although monocytes can also express MPO). At each endpoint, additional biopsies (6 mm) are harvested and stored at −70° C. As previously performed in the Yager laboratory, harvested tissues are extracted and MPO activity determined as a function of tissue weight or of total protein. Antioxidant status is determined using commercial kits.

### Example XXXV

### Stability of Silver Formulations

The stability and effectiveness of mafeninde acetate gel, silver sulfadiazine cream, polystyrene sulfonate silver gel with glutathione, silver sulfadiazine with glutathione and xanthine-based silver carbene with glutathione were formulated aseptically and packaged in airtight glass vials. Nine aliquots were made for each formulation and eight were placed in a 40° C. oven minus an aliquot taken for baseline. Samples were removed every two weeks for sixteen weeks and stored in a −80° C. freezer to allow for concurrent sample analysis. This procedure represents a quadrupling of the rate of any degradation process.

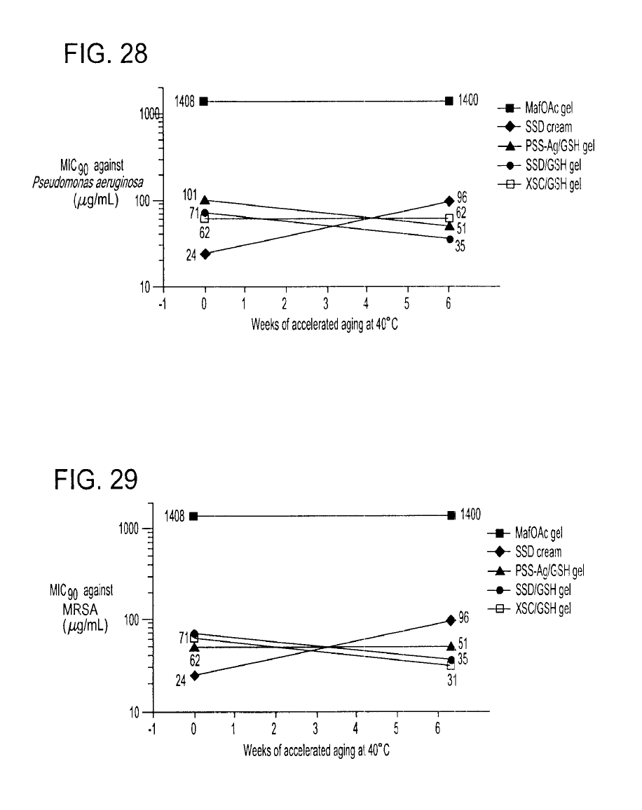

Samples of agar were doped with specific concentrations of the accelerated aging samples to form a gradient of formulation concentration. These samples were then poured into sterile 6-well tissue culture plates and allowed to harden. The agar filled wells were inoculated with 10⁴ colony forming units (C FUs) of bacteria, either Pseudomonas aeruginosa (PA) or Methicillin resistant Staphylococcus Aureus (MRSA) diluted in 100 μL of tryptic soy broth supplemented with 10% fetal bovine serum, and allowed to incubate overnight (16-18 hours). The well plates were examined post incubation and colonies were counted. The OD600 nm was monitored until OD600 nm of untreated control reaches an OD600 nm of approximately 1.0. The MIC₉₀ was identified as the well with the lowest concentration of formulation that had <10% (<100) of the colonies added and shown in FIGS. 28 and 29.

### Example XXXVI

### Procedure for IC₅₀ (Biocompatibility) Assays

The toxicity of mafeninde acetate gel, silver sulfadiazine cream, polystyrene sulfonate silver gel with glutathione, silver sulfadiazine with glutathione and xanthine-based silver carbene with glutathione were formulated aseptically and packaged in airtight glass vials. Nine aliquots were made for each formulation and eight were placed in a 40° C. oven minus an aliquot taken for baseline. Samples were removed every two weeks for sixteen weeks and stored in a −80° C. freezer to allow for concurrent sample analysis. This procedure represents a quadrupling of the rate of any degradation process.

Human neonatal fibroblasts grown in 10% FBS medium (Dulbecco's modification of eagle's medium supplemented with 10% fetal bovine serum) to confluence. These cells were released with a 0.25% trypsin solution, washed with 10% FBS medium, concentrated, and seeded onto 96-well sterile plasma treated polystyrene tissue culture plates at a density of 10,000 cells per well. The plates were allowed to incubate overnight in at 37° C. in 95% relative humidity with 5% CO₂. Following incubation, the medium was removed from the cells and medium doped with specific concentrations of mafeninde acetate gel, silver sulfadiazine cream, poly styrene sulfonate silver gel with glutathione, silver sulfadiazine with glutathione and xanthine-based silver carbene with glutathione were added to separate wells to create a gradient of formulation concentration. The plates were allowed to incubate overnight in at 37° C. in 95% relative humidity with 5% CO₂. Following incubation with the formulation doped media, an appropriate volume of CellTiter 96® Aqueous One Solution Cell Proliferation Assay solution (Promega, Madison, Wis.) was added and allowed to incubate for 4 hours at 37° C. in 95% relative humidity with 5% CO₂. Following incubation, the plates were evaluated for absorbance at 490ₙₘ with a BioTek Synergy H1 plate reader. The IC₅₀ as shown in FIG. 27 was identified as the well with the highest concentration of formulation that had >50% cell viability (calculated from absorbance at 490ₙₘ, where higher absorbance indicates higher viability).

Those skilled in the art will recognize that numerous modifications and changes may be made to the foregoing exemplary embodiment according to the comprehensive and detailed teachings herein, without departing from the scope of the claimed invention. Equivalent modifications of the invention, in its various aspects, will be obtainable by those skilled in the art following the teachings herein, without undue experimentation, which should not detract from the pioneering scope of Applicants' discoveries. No single feature, function or property of the exemplary embodiments may be considered essential without comprehension of the whole invention, whereby the scope of the invention should not be limited by any particular embodiments herein described but should be defined only by the appended claims.

We claim:

1. An antimicrobial composition comprising an oligodynamic metal and a thiol compound, wherein the thiol compound decreases toxicity of the oligodynamic metal.

2. The composition of claim 1, wherein the oligodynamic metal is copper, silver, zinc, or bismuth.

3. The composition of claim 1, wherein the oligodynamic metal comprises at least 10% of the composition.

4. The composition of claim 1, wherein the oligodynamic metal comprises at least 20% of the compositions.

5. The composition of claim 1, wherein the oligodynamic metal comprises at least 30% of the composition.

6. The composition of claim 1, wherein the thiol compound is glutathione, penicillamine, bacillithiol, mycothiol, cysteine, or 4-mercaptophenylboronic acid.

7. The composition of claim 1 further comprising one or more secondary therapeutic agents.

8. The composition of claim 7, wherein the secondary therapeutic agent is an antimicrobial, antiseptic, antifungal, growth factor, antioxidant, anesthetic or analgesic agent.

9. The composition of claim 1 formulated for topical administration.

10. The composition of claim 1, wherein the thiol compound increases solubility of the oligodynamic metal.

11. An antimicrobial composition comprising an oligodynamic metal and a cationic carrier; wherein the cationic carrier is carboxymethyl cellulose, alginic acid, polyacrylic acid or carboxylate.

12. The composition of claim 11, wherein the oligodynamic metal is copper, silver, zinc, or bismuth.

13. The composition of claim 11, wherein the oligodynamic metal comprises at least 10% of the composition.

14. The composition of claim 11, wherein the oligodynamic metal comprises at least 20% of the compositions.

15. The composition of claim 11, wherein the oligodynamic metal comprises at least 30% of the composition.

16. The composition of claim 11 further comprising one or more secondary therapeutic agents.

17. The composition of claim 16, wherein the secondary therapeutic agent is an antimicrobial, antiseptic, antifungal, growth factor, antioxidant, anesthetic or analgesic agent.

18. An antimicrobial composition comprising an oligodynamic metal, a polysulfonated compound, and a thiol compound, wherein the composition has anti-proteolytic and cytokine protective activity.

19. The antimicrobial composition of claim 18, wherein the oligodynamic metal is copper, silver, zinc, or bismuth.

20. The composition of claim 18, wherein the thiol compound is glutathione.

21. The composition of claim 18, wherein the thiol compound is penicillamine, bacillithiol, mycothiol, cysteine or 4-mercaptophnylboronic acid.

22. The composition of claim 18, wherein the oligodynamic metal comprises at least 10% of the composition.

23. The composition of claim 18, wherein the oligodynamic metal comprises at least 20% of the composition.

24. The composition of claim 18, wherein the oligodynamic metal comprises at least 30% of the composition.

25. The composition of claim 18, wherein the polysulfonate is heparin sulfate, chondroitin sulfate, dermatan sulfate, keratin sulfate or aggrecan sulfate.

26. The composition of claim 18 further comprising one or more secondary therapeutic agents.

27. The composition of claim 26, wherein the secondary therapeutic agent is an antimicrobial, antiseptic, antifungal, growth factor, antioxidant, anesthetic or analgesic agent.

28. An antimicrobial composition comprising a polysulfonated compound and a quaternary ammonium compound, wherein the composition has anti-proteolytic and cytokine protective activity.

29. The composition of claim 28, wherein the quaternary ammonium compound is selected from alkyl ammonium halides, alkyl aryl ammonium halides, octyl phenoxy ethoxy ethyl dimethyl benzyl ammonium chloride, N-(laurylcocoaminoformylmethyl)-pyridinium chloride, lauryloxyphenyltrimethyl ammonium chloride, cetylaminophenyltrimethyl ammonium methosulfate, dodecylphenyltrimethyl ammonium methosulfate, dodecylbenzyltrimethyl ammonium chloride, chlorinated or dodecylbenzyltrimethyl ammonium chloride.

30. The composition of claim 28, wherein the polysulfonate compound is selected from a polysaccharide, polyvinyl sulfate, poly acrylamidomethyl propane sulfate, poly methyl styrene sulfonate, or poly(methyl styrene sulfonate)-co-(polymethylmethacrylate).

31. The composition of claim 28, wherein the polysulfonate is heparin sulfate, chondroitin sulfate, dermatan sulfate, keratin sulfate or aggrecan sulfate.

32. The composition of claim 28 further comprising one or more secondary therapeutic agents.

33. The composition of claim 32, wherein the secondary therapeutic agent is an antimicrobial, antiseptic, antifungal, growth factor, antioxidant, anesthetic or analgesic agent.

34. An antimicrobial composition comprising a polysulfonated compound and chlorhexidine wherein the composition has antiseptic, anti-proteolytic and cytokine protective activity.

35. The composition of claim 34, wherein the polysulfonate compounds is selected from a polysaccharide, polyvinyl sulfate, poly acrylamidomethyl propane sulfate, poly methyl styrene sulfonate, or poly(methyl styrene sulfonate)-co-(polymethylmethacrylate).

36. The composition of claim 34, wherein the polysulfonate is heparin sulfate, chondroitin sulfate, dermatan sulfate, keratin sulfate or aggrecan sulfate.

37. The composition of claim 34 further comprising one or more secondary therapeutic agents.

38. The composition of claim 37, wherein the secondary therapeutic agent is an antimicrobial, antiseptic, antifungal, growth factor, antioxidant, anesthetic or analgesic agent.

39. An antimicrobial composition comprising a polysulfonated compound combined with octenidine wherein the composition has antiseptic, anti-proteolytic and cytokine protective activity.

40. The composition of claim 39, wherein the polysulfonate compounds is selected from a polysaccharide, polyvinyl sulfate, poly acrylamidomethyl propane sulfate, poly methyl styrene sulfonate, or poly(methyl styrene sulfonate)-co-(polymethylmethacrylate).

41. The composition of claim 39, wherein the polysulfonate is heparin sulfate, chondroitin sulfate, dermatan sulfate, keratin sulfate or aggrecan sulfate.

42. The composition of claim 39 further comprising one or more secondary therapeutic agents.

43. The composition of claim 42, wherein the secondary therapeutic agent is an antimicrobial, antiseptic, antifungal, growth factor, antioxidant, anesthetic or analgesic agent.

44. A method of treating a wound exhibiting an initial, elevated protease response comprising:
a. administering a first antimicrobial composition with high anti-protease activity, comprising an oligodynamic metal coupled to a sulfonated compound;
b. removing the first composition with high-protease activity when the wound advances to exhibit less than the initial level of protease activity; and
c. administering a second, different composition comprising an antimicrobial compound having less or no protease activity in comparison to the first composition.

45. The method of claim 44, further comprising administering an antiseptic with the first or second composition.

46. The method of claim 44, wherein the antimicrobial compound is silver sulfadiazine cream.

47. The method of claim 44, wherein the oligodynamic metal is copper, silver, zinc, or bismuth.

48. The composition of claim 44, wherein the oligodynamic metal comprises at least 10% of the composition.

49. The composition of claim 44, wherein the oligodynamic metal comprises at least 20% of the composition.

50. The composition of claim 44, wherein the oligodynamic metal comprises at least 30% of the composition.

51. The method of claim 44, further comprising administering an antibiotic with the first or second composition.

52. The method of claim 44, further comprising administering an analgesic with the first or second composition.

53. The method of claim 44, further comprising administering an anesthetic with the first or second composition.

54. The method of claim 44, further comprising administering a second antimicrobial compound with the first or second composition.

55. The method of claim 44, further comprising skin grafting.

56. The method of claim 44, wherein the first composition further comprises a thiol compound.

57. The method of claim 56, wherein the thiol compound is glutathione.

58. The method of claim 44, wherein the first composition is water soluble.

59. The method of claim 58, wherein the first composition is insoluble in deionized water.

60. A method of treating wounds with abnormal protease levels comprising:
a. detecting a clinically elevated protease level in the wound;
b. administering a sulfonated antimicrobial compound possessing high antiproteolytic activity; and
c. re-measuring a protease levels in the wound;
wherein when protease levels are substantially reduced toward normal for wound healing, the sulfonated compound is removed and replaced with a non-sulfonated antimicrobial compound having less or no antiproteolytic activity compared to the first antimicrobial compound.

61. The method of claim 60, further comprising administering an antiseptic with the sulfonated compound or the non-sulfonated antimicrobial compound.

62. The method of claim 60, further comprising administering an antibiotic with the sulfonated compound or the non-sulfonated antimicrobial compound.

63. The method of claim 60, further comprising administering an analgesic with the sulfonated compound or the non-sulfonated antimicrobial compound.

64. The method of claim 60, further comprising administering an anesthetic with the sulfonated compound or the non-sulfonated antimicrobial compound.

65. The method of claim 60, further comprising administering a second antimicrobial with the sulfonated compound or the non-sulfonated antimicrobial compound.

66. The method of claim 60, further comprising skin grafting.

67. The method of claim 60, wherein the sulfonated compound further comprises a thiol compound.

68. The method of claim 67, wherein the thiol compound is glutathione.

69. The method of claim 60, wherein the sulfonated compound is water soluble.

70. The method of claim 69, wherein the sulfonated compound is insoluble in deionized water.

71. A method of treating a wound infected with bacteria comprising administering an antimicrobial composition comprising an oligodynamic metal combined with a polysulfonated compound and a thiol compound, wherein the composition has anti-proteolytic and cytokine protective activity.

72. The method of claim 71, wherein the oligodynamic metal is copper, silver, zinc, or bismuth.

73. The composition of claim 71, wherein the oligodynamic metal comprises at least 10% of the composition.

74. The composition of claim 71, wherein the oligodynamic metal comprises at least 20% of the compositions.

75. The composition of claim 71, wherein the oligodynamic metal comprises at least 30% of the composition.

76. The method of claim 71, wherein the thiol compound is glutathione.

77. The method of claim 71, wherein the thiol compound is penicillamine, bacillithiol, mycothiol, cysteine or 4-mercaptophnylboronic acid.

78. The method of claim 71, wherein the polysulfonate compound is selected from a polysaccharide, polyvinyl sulfate, poly acrylamidomethyl propane sulfate, poly methyl styrene sulfonate, or poly(methyl styrene sulfonate)-co-(polymethylmethacrylate).

79. The method of claim 71, wherein the polysulfonate is heparin sulfate, chondroitin sulfate, dermatan sulfate, keratin sulfate or aggrecan sulfate.

80. The method of claim 71 further comprising a secondary therapeutic agent.

81. The method of claim 80, wherein the secondary therapeutic agent is an antimicrobial, antiseptic, antifungal, growth factor, antioxidant, anesthetic or analgesic agent.

82. A method of treating a wound colonized by bacteria with an effective amount of an anti-microbial silver composition without reducing endogenous glutathione reserves, wherein the composition includes a thiol, and wherein exposure of the wound to the anti-microbial silver compound is followed by maintenance of glutathione or glutathione transferase levels in a wound exudate that is at least 20% higher than glutathione or glutathione transferase levels observed following treatment with the same or different antimicrobial silver compound in the absence of added thiol.

83. The method of claim 79, wherein the thiol compound is glutathione.

84. The method of claim 80, wherein the thiol compound is selected from penicillamine, bacillithiol, mycothiol, cysteine, or 4-mercaptophenylboronic acid.

85. The method of claim 82, wherein the anti-microbial silver compound further comprises a sulfonated compound.

86. The method of claim 82, wherein the sulfonated compound is a polysulfonate.

87. The method of claim 82, wherein the polysulfonate compound is selected from a polysaccharide, polyvinyl sulfate, poly acrylamidomethyl propane sulfate, poly methyl styrene sulfonate, or poly(methyl styrene sulfonate)-co-(polymethylmethacrylate).

88. The method of claim 82 wherein the polysulfonate is heparin sulfate, chondroitin sulfate, dermatan sulfate, keratin sulfate or aggrecan sulfate.

89. The method of claim 82, further comprising a secondary therapeutic agent.

90. The method of claim 89, wherein the secondary therapeutic agent is an antimicrobial, antiseptic, antifungal, growth factor, antioxidant, anesthetic or analgesic agent.

91. A composition comprising a solid form antimicrobial wound dressing material that turns to liquid when hydrated with an ionic solution but remains in solid form when contacted with a non-ionic solution, wherein the composition contains an effective antimicrobial compound.

92. The composition of claim 91, wherein the effective antimicrobial compound is an oligodynamic metal.

93. The composition of claim 92, wherein the oligodynamic metal is selected from copper, silver, zinc, or bismuth.

94. The composition of claim 91 further comprising a thiol compound.

95. The solid form antimicrobial of claim 94, wherein the thiol compound is selected from glutathione, penicillamine, bacillithiol, mycothiol, cysteine, or 4-mercaptophenylboronic acid.

96. The composition of claim 91 further comprising a sulfonated compound.

97. The composition of claim 96, wherein the sulfonated compound is a polysulfonate compound selected from a polysaccharide, polyvinyl sulfate, poly acrylamidomethyl propane sulfate, poly methyl styrene sulfonate, or poly(methyl styrene sulfonate)-co-(polymethylmethacrylate).

98. The composition of claim 96, wherein the sulfonated compound is a polysulfonate selected from heparin sulfate, chondroitin sulfate, dermatan sulfate, keratin sulfate or aggrecan sulfate.

99. The composition of claim 92, wherein the oligodynamic metal comprises at least 10% of the composition.

100. The composition of claim 92, wherein the oligodynamic metal comprises at least 20% of the compositions.

101. The composition of claim 92, wherein the oligodynamic metal comprises 30% of the composition.

102. A method of producing a salt of a polysulfonated compound comprising:
a. dissolving a polysulfonated compound in deoxygenated deionized water,
b. combining a sulfonated compound with a mass of acetate salt sufficient to obtain a quantity of the desired cation;
c. stirring the solution;
d. adding the solution to a volume of concentrated alcohol, precipitating the polymer; and
e. filtering and washing the precipitated polymer.

103. The method of claim 102, wherein the polysulfonated compound is poly(4-styrenesulfonate sodium).

104. The method of claim 102, wherein the acetate salt is silver acetate, zinc acetate, copper acetate, mafenide acetate, chlorhexidine diacetate, or octenidine dihydrochloride.

105. A biomedical wound therapy device comprising an antimicrobial composition according to claim 1 invested at a surface of a bandage, covering, coating, film or dressing adapted to be placed in contact with an exposed wound surface of a mammalian subject.

106. The device of claim 105, comprising a biogel film, patch, or dressing.

107. The device of claim 102, wherein the antimicrobial composition is coated on or invested within an exposed surface of a polymer or textile wound dressing, bandage or covering.

108. The device of claim 102, wherein the antimicrobial composition is coated on or invested within an exposed surface of a wound dressing material selected from gauze, films, absorbtives, tapes, wraps, bandages, hydrocolloids, hydrogels, alginates and collagen wound dressings.

109. The device of claim 102, wherein the antimicrobial composition comprises an oligodynamic metal combined with a thiol, and wherein use of the device for prolonged periods minimizes discoloration and long term cosmetic damage in comparison to other olidgodynamic metal invested wound dressings.
